(19) United States
(12) Patent Application Publication
(10) Pub. No.: US 2001/0036929 A1
(43) Pub. Date: Nov. 1, 2001

(54) Xrcc3 is required for assembly of Rad51-complexes in vivo

(75) Inventors: Ralph R. Weichselbaum, Chicago, IL (US); Douglas K. Bishop, Chicago, IL (US)

Correspondence Address: Steven L. Highlander, Esq., FULBRIGHT & JAWORSKI L.L.P., Suite 2400, 600 Congress Avenue, Austin, TX, 78701, US

(73) Assignee: ARCH Development Corporation.

(21) Appl. No.: 09/844,538

(22) Filed: Apr. 26, 2001

Related U.S. Application Data
(62) Division of application No. 09/404,053, filed on Sep. 22, 1999.
(60) Provisional application No. 60/101,909, filed on Sep. 25, 1998.

(51) Int. Cl. A61K 48/00; A61K 33/24

(52) U.S. Cl. 514/44; 514/34; 424/649

(57) ABSTRACT

The present invention relates to the interaction of Rad51 and Xrcc3 to form a complex that mediates DNA repair in eukaryotic cells. A functional Rad51/Xrcc3 complex can be introduced into a cell to increase the resistance of the cell to DNA damaging agents. The invention also provides for a clinical application of a regimen combining Rad51 and Xrcc3 to reduce the side effects of radiotherapy and chemotherapy in a patient. In addition, the invention discloses methods for identifying candidate substances that interact with the Rad51/Xrcc3 complex. In another embodiment of the invention, preventing the formation of the Rad51/Xrcc3 complex increases the susceptibility of a cell to DNA damaging agents. This strategy can be used in combination with a DNA damaging agent or factor to kill cancerous cells.

### BACKGROUND OF THE INVENTION

1. Field of the Invention

The present invention relates generally to the field of DNA damage repair. More particularly, it provides a method for altering the sensitivity of a cell to DNA damaging agents, including radiation and chemotherapeutic agents.

2. Description of Related Art

Although significant efforts continue towards the development of effective anti-cancer strategies, many prevalent forms of human cancer still resist effective chemotherapeutic intervention, as well as radiotherapy. Chemotherapy involves the use of chemotherapeutic agents to treat a wide variety of cancers. Radiotherapy uses x-rays and gamma-irradiation to treat cancer, as well as other non-malignant diseases. Radiotherapy may be the only treatment used in some cases, but it also may be used in conjunction with surgery, chemotherapy, or both. The best treatment regimen, whether it be surgery, radiotherapy, chemotherapy, or a combination of these methods, seeks to kill all of the so-called “clonogenic” malignant cells to prevent regrowth of the tumor mass.

Certain types of tumors are more amenable than others to therapies involving therapeutic agents and regimens. For example, lymphomas, and tumors of the blood and blood-forming organs, e.g., leukemias, have generally been more responsive to chemotherapeutic therapy, while solid tumors, such as carcinomas, generally prove to be more resistant to such therapies.

One underlying reason for this phenomenon may be that blood-based tumors are physically more accessible to chemotherapeutic agents, whereas it is often difficult for most chemotherapeutic agents to reach all of the cells of a solid tumor. However, increasing the dose of chemotherapeutic agents in order to achieve the desired effect most often results in toxic side effects that limit the effectiveness of chemotherapy.

Even immunotoxins that are directed to selected cancer cell antigens have proven to be of limited use in the treatment of solid tumors (Weiner et al., 1989; Byers et al., 1989). One reason for this is that solid tumors are generally impermeable to antibody-sized molecules, often exhibiting specific uptake values of less than 0.001% of the injected dose/g of tumor in human studies (Sands et al., 1988; Epenetos et al., 1986).

Further significant problems that can apply to any conventional chemotherapeutic agent include the formation of mutants that escape cell killing and regrow; the dense packing of cells within the tumor that creates a physical barrier to macromolecular transport; the absence of lymphatic drainage, creating an elevated interstitial pressure that reduces extravasation and fluid convection; the heterogeneous distribution of blood vessels that leaves certain tumor cells at a considerable diffusion distance; and the adsorption of agents in the perivascular tumor cells. Radiotherapy can overcome many of the shortcomings of chemotherapy. However, radiation suffers from a lack of target specificity, and it also can cause serious side effects.

Some of the toxic side effects of both chemotherapeutic agents and radiation treatments are the result of cellular DNA damage in healthy cells. The Rad51 protein is of particular interest in the study of cellular response to DNA damage because of its structural and functional similarity to the E. coli RecA protein (Shinohara and Ogawa, 1995). RecA is known to play a central role in the prokaryotic response to DNA damage (Roca and Cox, 1991; Kowalczykowski et al., 1994; Friedberg et al., 1995). The functional form of RecA and Rad51 is a multimeric helical nucleoprotein filament. RecA nucleoprotein filaments are rapidly assembled on single-stranded DNA segments that form after various types of DNA damage.

Rad51 is a conserved member of a family of eukaryotic proteins related to RecA. Additional family members have been identified in a wide range of eukaryotes including yeasts and mammals (Stassen et al., 1997). The completion of the Saccharomyces cerevisiae genomic sequence established that four previously-identified proteins, Rad51, Rad55, Rad57, and Dmc1, constitute the complete set of RecA relatives in this organism (Aboussekhra et al., 1992; Basile et al., 1992; Lovett, 1994; Bishop et al., 1992; Shinohara et al., 1992). Thus far, seven members of the Rad51 protein family been identified in mammals. These include: highly conserved human Rad51, HsRAD51 (Shinohara et al., 1993), murine and human Dmc1 (Sato et al., 1995; Habu et al., 1996), human XRCC2 (Liu et al., 1995) and XRCC3 (Thompson et al., 1996), which were isolated by their ability to complement hamster mutant cells, and finally RAD51B (Albala et al., 1997), RAD51C (Dosanjh et al., 1998), and RAD51D, which were found by database searching.

Saccharomyces cerevisiae Rad51 (ScRad51) has been shown to be functionally similar to RecA. Both RecA and ScRad51 promote homology-dependent repair of DNA double-strand breaks induced by ionizing radiation (Krasin and Hutchinson, 1977; Jachymczyk et al., 1981). Homology-dependent double-strand break repair is an accurate process that usually repairs double-strand breaks (DSBs) without generating insertions, deletions, or other chromosomal rearrangements. This accuracy results from the use of an undamaged copy of the broken DNA as a template for the repair process (Stahl, 1994). The recruitment of an undamaged copy of the DNA requires strand exchange activity. Like RecA (Cox and Lehman, 1981) and ScRad51 (Sung, 1994), HsRAD51 promotes DNA strand exchange in vitro (Baumann et al., 1996; Gupta et al., 1997), indicating that the role of Rad51 in recombinational DSB repair is likely to be conserved in mammals. Acquisition of genetic evidence implicating mammalian RAD51 genes in DNA repair has been complicated by the fact that rad51 mutant mice die during early embryonic development (Lim and Hasty, 1996; Tsuzuki et al., 1996). However, studies on early mouse embryos suggest that the murine rad51 mutant, like its yeast counterpart, is sensitive to ionizing radiation (Lim and Hasty, 1996).

In addition to being able to repair DSBs by homology-dependent mechanisms, eukaryotes also have a homology-independent end-joining. In mammalian cells, the homology independent process is the predominant pathway for the repair of damage-induced double-strand breaks. DNA protein kinase (DNA-PK), a heterotrimeric protein with both protein kinase and DNA end-binding activity, is one of several proteins that promote homology-independent repair of DSBs (Anderson and Lees-Miller, 1992; Gottlieb and Jackson, 1993). Mice with the scid mutation are defective in the catalytic subunit of DNA-PK (Kirchgessner et al., 1995; Blunt et al., 1996). The Chinese hamster ovary (CHO) cell line xrs5 is one of several mutants defective in Ku86 protein, a subunit required for the end binding activity of DNA-PK which is encoded by the XRCC5 gene (Errami et al., 1996; Smider et al., 1994; Ross et al., 1995; Taccioli et al., 1994).

Immunostaining of both yeast and human nuclei with antibodies against RecA homologues has shown that they form visible complexes during meiotic recombination and mitotic DNA repair (Bishop, 1994; Haaf et al., 1995; Ashley et al., 1995; Terasawa et al., 1995; Li et al., 1996). These visible complexes include Rad51 foci, which form in mammalian fibroblasts and lymphocytes in response to ionizing and ultraviolet radiation, as well as the DNA alkylating agent methylmethane sulfonate, MMS (Haaf et al., 1995). Formation of subnuclear Rad51 foci also are induced by the DNA crosslinking agent cisplatin. The observation that Rad51 foci are found in a variety of cells undergoing DNA repair and recombination suggests that these foci mark nucleoprotein complexes engaged in recombinational repair.

The possibility that Rad51 foci seen in CHO cells correspond to sites of DNA damage is supported by observations made in normal meiotic yeast cells (Bishop, 1994). First, ScRad51 and ScDmc1, two RecA homologs known to be required for repair of meiotic DSBs, colocalize with one another in wildtype cells. This is an expected result if the foci mark sites where multiple recombination proteins are engaged in recombination. Second, the foci formed by RecA homologs appear specifically at the time of meiotic DSB repair. Third, mutational analysis shows that blocking DSB formation blocks the appearance of ScRad51 foci and that blocking DSB repair blocks the disappearance of foci. These results support the hypothesis that foci of RecA-like proteins mark sites of ongoing recombination and repair. A very recent study demonstrated that ultra-soft X-rays, which were used to damage subsections of nuclei, did not evidence localization of Rad51 foci to damaged regions, raising the possibility that assembly is not specific to damaged sites (Nelms et al., 1998). However, it is possible that some or all of the Rad51 foci seen in these studies were not induced by radiation, but were instead the S-phase foci observed in untreated fibroblasts (Haaf, 1995).

Taken together, these findings implicate a role for Rad51 in DNA damage repair in response to DNA damaging agents. A significant need exists for the development of novel strategies to regulate Rad51-mediated DNA damage repair in response to both radiotherapy and chemotherapeutic agents.

### SUMMARY OF THE INVENTION

The present invention provides methods to increase cellular resistance, or enhance cellular susceptibility, to DNA damaging agents. In one aspect, the present invention is based on the observation that the formation of damage-induced Rad51 foci does not occur in the CHO cell line irs1SF, which is sensitive to DNA damaging agents. The inventors demonstrate that the Rad51 focus formation defect of irs1SF cells is corrected by a construct that encodes the repair protein Xrcc3. These results suggest that Xrcc3 is required for the assembly or stabilization of a multimeric form of Rad51 during DNA repair. The inventors propose that Rad51 and Xrcc3 interact to form a complex that facilitates DNA repair. Delivery of the Rad51/Xrcc3 complex to an animal patient will alter the relationship of DNA repair to both health and disease. The Rad51/Xrcc3 complex also provides for the identification of additional factors which effect the susceptibility of cells to DNA damaging agents.

Thus, in a first embodiment, the present invention concerns methods for preventing or treating DNA damage in cells exposed to DNA damaging agents. One aspect of the present invention contemplates a method of producing a functional Rad51/Xrcc3 complex comprising providing to a cell a first polynucleotide encoding a Rad51 polypeptide; a second nucleic acid encoding a Xrcc3 polypeptide; and expressing the complex in a cell, wherein the coexpression of the polypeptides allows for the formation of a functional Rad51/Xrcc3 complex. In one embodiment, the first and second polynucleotides can be provided to a cell as naked DNA. In still another embodiment, the first and second polynucleotides are provided to a cell through liposomal delivery, or alternatively, viral delivery.

In another aspect, the first and second polynucleotides are contained in different expression constructs, and both are under the control of a first and a second promoter, respectively. In one embodiment, expression of the first and second polynucleotides is controlled by a radiation-inducible promoter. Choices for an ionizing radiation-inducible promoter include a CArG domain of an Egr-1 promoter, a los promoter, a c-jun promoter, or a TNF-α promoter. In a further embodiment, the polynucleotides comprise a polyadenylation signal positioned 3′ to the first and second polynucleotides, respectively. In yet another embodiment, the expression constructs contain a selectable marker. In other aspects of this embodiment, the expression constructs may be defined as viral vectors.

In yet another embodiment of the invention, the first and second polynucleotides are contained in the same expression construct and are under the control of a first and a second promoter, respectively. In one embodiment, the first and second polynucleotides are controlled by a radiation-inducible promoter. It is contemplated in certain embodiments of the present invention that the expression construct may further comprise a first polyadenylation signal positioned 3′ to the first polynucleotide and a second polyadenylation signal positioned 3′ to the second polynucleotide. In another aspect, the expression construct contains a selectable marker. The expression construct may also be defined as a viral vector.

In other embodiments, the expression construct further comprises both the first and second polynucleotides under the control of a first promoter. In one aspect, the first promoter is controlled by a radiation-inducible promoter. In certain embodiments, the expression construct may further comprise a polyadenylation signal positioned 3′ to the second polynucleotide. In another aspect, the first and second polynucleotides are expressed as a fusion protein.

In a second embodiment, the present invention concerns methods for enhancing the susceptibility of cells to DNA damaging agents, by inhibiting the formation of a functional Rad51/Xrcc3 complex, comprising the steps of (i) providing to a cell a polynucleotide encoding a Xrcc3 antisense RNA; and (ii) expressing the Xrcc3 antisense RNA in a cell, wherein the expression of said Xrcc3 antisense RNA blocks the expression of endogenous Xrcc3, thereby preventing the formation of a functional Rad51/Xrcc3 complex. In one embodiment, the polynucleotide is provided to a cell as naked DNA. In another embodiment, the polynucleotide is provided to a cell through liposomal delivery, or alternatively, viral delivery. In one aspect, the polynucleotide is contained in an expression construct under the control of a promoter. In another aspect, the polynucleotide is controlled by a radiation-inducible promoter. In a further embodiment, the expression construct has a polyadenylation signal positioned 3′ to the polynucleotide. In yet another embodiment, the expression construct contains a selectable marker. The expression construct may also be defined as a viral vector.

In other embodiments of the present invention, Xrcc3 antisense RNA blocks the expression of endogenous Xrcc3 in a cell by binding to the promoter, exon sequences, intron sequences, exon-intron splice junctions, or transcription start site of the Xrcc3 gene. Alternatively, Xrcc3 antisense RNA blocks expression of endogenous Xrcc3 by binding to the translation start site or ribosomal binding site of Xrcc3 mRNA.

Also contemplated by the present invention is a method of identifying a modulator of Rad51/Xrcc3 complex formation comprising the steps of (a) providing a Rad51 and a Xrcc3 under conditions suitable for Rad51/Xrcc3 complex formation; (b) contacting the components of step (a) with a candidate substance; and (c) determining the effect of said candidate substance on Rad51/Xrcc3 complex formation, wherein an increase or decrease in Rad51/Xrcc3 complex formation, as compared to Rad51/Xrcc3 complex formation in the absence of the same candidate substance, identifies the candidate substance as a modulator of Rad51/Xrcc3 complex formation. In particular embodiments, the conditions suitable for Rad51/Xrcc3 complex formation is exposure to DNA damage. The DNA damage may result from ionizing radiation, ultraviolet radiation, cisplatin, adriamycin, 5-fluorouracil, etoposide, camptothecin, actinomycin-D, mitomycin C, verapamil or the DNA alkylating agent methylmethane sulfonate (MMS).

It is contemplated that the candidate substance may be identified by utilizing the Rad51/Xrcc3 complex in a yeast two-hybrid system or a co-immunoprecipitation assay. In particular embodiments, the candidate substance is a polynucleotide, a polypeptide, or a small molecule inhibitor. In one embodiment, the polynucleotide encodes, or the polypeptide is, an enzyme, an antibody, an antisense mRNA, or a transcription factor. In another embodiment, the antibody reacts immunologically to the Rad51/Xrcc3 complex. In yet another embodiment, the polynucleotide is an expression construct comprising a promoter active in eukaryotic cells. The candidate substance may also be selected from a small molecule or peptide library.

The invention also concerns methods for preventing or treating cellular damage in an animal or human patient exposed to DNA damaging agents, or one that is accidentally exposed to DNA damaging agents. These methods generally comprise administering to the animal or human patient a pharmaceutically acceptable composition comprising Rad51 or Xrcc3. The pharmaceutically acceptable composition may be provided to the patient as a first polynucleotide encoding a Rad51 polypeptide or a second polynucleotide encoding a Xrcc3 polypeptide. A polynucleotide may be delivered to the animal patient as naked DNA or through viral delivery.

In a further aspect of the invention, the polynucleotides are under the control of a promoter operatively linked to the first and second polynucleotides, respectively. In one embodiment, the promoter is a radiation-inducible promoter. In another embodiment, the polynucleotides have a polyadenylation signal positioned 3′ to the first and second polynucleotides, respectively. Rad51 or Xrcc3 also may be provided to the animal patient under the control of a selectable marker. In yet another embodiment, the polynucleotide is contained in a viral vector.

It also is contemplated that both Rad51 and Xrcc3 are provided to said animal or human patient as polynucleotides encoding a Rad51 polypeptide and a Xrcc3 polypeptide, respectively. The polynucleotides may be delivered to the animal patient as naked DNA or through viral delivery. In yet another embodiment, the polynucleotides are contained in viral vectors. Rad51 and Xrcc3 may also be provided to the animal patient under the control of a selectable marker. In another embodiment, Rad51 and Xrcc3 are provided to the animal patient as a Rad51/Xrcc3 protein complex.

The invention further contemplates that the DNA damaging agent causing cellular damage to the animal patient is radiation or a chemotherapeutic agent. In one embodiment, the radiation is either ionizing radiation or ultraviolet radiation. In another embodiment, the chemotherapeutic agent includes such classical chemotherapeutic agents as cisplatin, adriamycin, 5-fluorouracil, etoposide, camptothecin, actinomycin-D, mitomycin C, verapamil or the DNA alkylating agent methylmethane sulfonate (MMS).

The present invention also proposes methods for treating an animal or human patient with cancer. These methods generally comprise contacting cancer cells in the animal or human patient with a pharmaceutically acceptable composition comprising Rad51 antisense RNA or Xrcc3 antisense RNA. The invention also contemplates contacting the cancer cells of the patient with a DNA damaging agent. The pharmaceutically acceptable composition may be provided to the patient as a first polynucleotide encoding a Rad51 antisense RNA or a second polynucleotide encoding a Xrcc3 antisense RNA. A polynucleotide may be delivered to the animal patient as naked DNA or through viral delivery.

In a further aspect of the invention, the polynucleotides are under the control of a promoter operatively linked to the first and second polynucleotides, respectively. In one embodiment, the promoter is a radiation-inducible promoter. In another embodiment, the polynucleotides have a polyadenylation signal positioned 3′ to the first and second polynucleotides, respectively. Rad51 antisense RNA or Xrcc3 antisense RNA also may be provided to the animal patient under the control of a selectable marker. In yet another embodiment, the polynucleotide is contained in a viral vector.

The invention also contemplates that both Rad51 antisense RNA and Xrcc3 antisense RNA are provided to said animal or human patient as polynucleotides encoding a Rad51 antisense RNA and a Xrcc3 antisense RNA, respectively. The polynucleotides may be delivered to the animal patient as naked DNA or through viral delivery. In yet another embodiment, the polynucleotides are contained in viral vectors. Rad51 antisense RNA and Xrcc3 antisense RNA may also be provided to the animal patient under the control of a selectable marker.

The invention further contemplates that the DNA damaging agent causing cellular damage to the cancer cells is radiation or a chemotherapeutic agent. In one embodiment, the radiation is either ionizing radiation or ultraviolet radiation. In another embodiment, the chemotherapeutic agent includes such classical chemotherapeutic agents as cisplatin, adriamycin, 5-fluorouracil, etoposide, camptothecin, actinomycin-D, mitomycin C, verapamil or the DNA alkylating agent methylmethane sulfonate (MMS).

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-FIG. 1F shows the X-ray dose response and time course analysis of radiation-induced Rad51 foci. FIG. 1A and FIG. 1B show X-ray dose response analysis of Rad51 focus formation in CHO cell lines. Samples were treated with the indicated doses of X-rays and then incubated for 3 hours before fixation and staining with a-Rad51 antibody. Images were taken of an unselected sample of 50 nuclei and the number of Rad51 foci in each nucleus was scored. In the plots shown, the area of the circles is proportional to the number of nuclei that displayed the quantity of foci indicated on the γ-axis. A numerical statistical display for each data set is shown in terms of mean, standard deviation in parentheses, and percentage of nuclei with <5 foci in square brackets. FIG. 1C-FIG. 1F show the time course analysis of the response in CHO cell lines. Cells were subjected to a dose of 9 Gy, incubated for the indicated times, fixed, stained, and analyzed as described for the dose response analysis.

FIG. 2 shows a Western blot analysis of Rad51 protein levels. Whole cell protein extracts were prepared from the cell lines indicated and subjected to Western blot analysis with a-HsRad51 antibody. 40 μg of total protein was loaded in each lane. The first lane contains 5 ng of purified recombinant HsRad51 protein (a positive control for antibody staining and a molecular weight standard). The next 8 lanes contain equal amounts of whole cell protein extracts from the four different cell lines indicated. The extracts were from cells that were unirradiated (−) or irradiated (+) to a dose of 9 Gy 3 hours prior to the time of extract preparation.

FIG. 3A-FIG. 3F shows that cisplatin induces Rad51 foci in AA8 cells, but not in irs1SF cells. FIG. 3A and FIG. 3B show the dose response of Rad51 foci to cisplatin treatment. CHO cells grown on culture plates were incubated in medium containing the indicated concentration of cisplatin for 1 hour, washed and incubated for an additional 3 hours prior to fixation. Fixed samples were immunostained with anti-HsRad51 antibody and examined as described in the legend of FIG. 1. Quantitation is as described in FIG. 1A legend. FIG. 3C-FIG. 3F show the time course analysis of cisplatin-induced Rad51 foci. Cells were incubated in medium containing cisplatin at a concentration of 10 μM for 1 hour, washed, and further incubated for the indicated time before fixation.

FIG. 4A-FIG. 4H shows that murine scid fibroblasts and CHO xrs5 cells form Rad51 foci in response to X-irradiation. Cells were treated with X-rays and then incubated for 3 hours before fixation and staining with a-Rad51 antibody. FIG. 4A and FIG. 4B show X-ray dose response analysis of Rad51 focus formation from a “normal” mouse fibrosarcoma cell-line 4102 and a scid mouse-derived fibroblast line. Quantitation is as described in FIG. 1A legend. FIG. 4C and FIG. 4D shows the time course analysis of Rad51 focus formation from 4102 and scid cells. FIG. 4E and FIG. 4F shows X-ray dose response analysis of Rad51 foci from the radiation-resistant CHO parent cell-line K1, and the radiation-sensitive derivative xrs5. FIG. 4G and FIG. 4H shows the time course analysis of Rad51 focus formation in CHO K1 and xrs5 cell-lines.

### DESCRIPTION OF ILLUSTRATIVE EMBODIMENTS

Radiotherapy and chemotherapy each have a limited capacity to treat many prevalent forms of human cancer. One problem with therapeutic regimens such as radiotherapy and chemotherapy is that while these treatments cause cellular damage and death in cancerous and diseased cells, they can also harm healthy host cells. Harmful secondary effects to the host organism can be severe, and are a limiting factor of the efficacy of these regimens. Reducing the toxicity of these therapeutic regimens will result in better options for therapeutic intervention, including potentially reducing the time course, dosage, and side effects of the regimens, all to the advantage of the animal patient. Therefore, the toxic side effects of radiotherapy or chemotherapy may be reduced if a strategy exists to enhance the susceptibility of diseased cells to DNA damaging agents, or alternatively, if cellular resistance to DNA damage in healthy cells is increased. A significant need exists for the development of novel strategies to regulate DNA damage repair in response to both radiotherapy and chemotherapeutic agents.

I. Present Invention

The E. coli RecA protein is known to play a central role in the prokaryotic response to DNA damage (Roca and Cox, 1991; Kowalczykowski et al., 1994; Friedberg et al., 1995). Rad51 is a conserved member of a family of eukaryotic proteins related to RecA, and therefore is of particular interest in the study of cellular response to DNA damage (Shinohara and Ogawa, 1995). Rad51 foci are visible complexes which form in mammalian fibroblasts and lymphocytes in response to ionizing and ultraviolet radiation, as well as to the DNA alkylating agent methylmethane sulfonate, MMS (Haaf et al., 1995). The DNA crosslinking agent cisplatin also induces formation of subnuclear Rad51 foci. The observation that Rad51 foci are found in a variety of cells undergoing DNA repair and recombination suggests that these foci mark nucleoprotein complexes engaged in recombinational repair.

The inventors demonstrate that Rad51 protein localizes to multiple subnuclear foci in CHO cells in response to ionizing radiation and treatment with the nucleotide crosslinking agent cisplatin. Formation of these foci likely reflects assembly of a multimeric form of Rad51 that promotes DNA repair. More importantly, the inventors show that Rad51 focus formation is defective in the radiation-sensitive CHO line irs1SF. Interestingly, both the Rad51 localization defect and the altered DNA damage resistance of irs1SF cells are rescued by DNA constructs that encode Xrcc3, a repair protein that is structurally related to Rad51 (Tebbs et al., 1995). The inventors also demonstrate that changes in the steady state level of Rad51 protein do not account for the irs1SF defect, nor do they account for the appearance of foci following DNA damage, indicating that the failure of irs1SF cells to form foci does not result from the failure of these cells to express Rad51 protein. Based on these observations, the inventors propose that a direct interaction between the Rad51 and Xrcc3 proteins is required for assembly or stabilization of Rad51 multimers at the sites of DNA damage in preparation for recombinational repair. These results suggest that Rad51/Xrcc3 complexes promotes cellular resistance to radiation, as well as other DNA damaging agents.

In a first embodiment, this invention provides a method of producing a functional Rad51/Xrcc3 complex in a cell. The use of the Rad51/Xrcc3 complex will alter the ability of a cell to repair DNA damage after exposure to a DNA damaging agent. A second embodiment of the invention involves preventing the formation of the Rad51/Xrcc3 complex by providing to a cell an antisense Xrcc3 mRNA transcript. This strategy will increase the susceptibility of a target cell to DNA damaging agents. A third embodiment of the invention involves identifying a candidate substance that modulates the formation and activity of the Rad51/Xrcc3 complex. Using the Rad51/Xrcc3 complex to screen for molecules that interact with the complex will allow for the identification of additional components that affect the susceptibility of cells to DNA damaging agents.

In another embodiment of the invention, Rad51, Xrcc3, or both Rad51 and Xrcc3, are administered to an animal patient to prevent or treat cellular damage that results from exposure to a DNA damaging agent. In an alternative embodiment of the present invention, Rad51 antisense RNA, Xrcc3 antisense RNA, or both antisense RNAs, are contacted with cancer cells in an animal patient to prevent the formation of the Rad51/Xrcc3 complex in the diseased cells. In a further embodiment, the cancer cells are then exposed to a DNA damaging agent. Since the cells will not have a functional Rad51/Xrcc3 complex to regulate DNA repair, they will be more susceptible to DNA damage and cell death. Included in the invention are treatments that involve cancers such as brain, lung, liver, spleen, kidney, lymph node, small intestine, pancreas, blood cells, colon, stomach, breast, endometrium, prostate, testicle, ovary, skin, head and neck, esophagus, bone marrow and blood tumors. The invention also is intended for use in benign neoplasms, including meningiomas, arteriovenous malformations, hemangiomas and the like. Cellular DNA damage may be caused by radiation or chemotherapeutic agents.

II. Polynucleotides

The present invention provides for, in one embodiment, delivery of Rad51 and Xrcc3 polynucleotides to a cell. Any reference to a polynucleotide should be read as encompassing a host cell containing that polynucleotide and, in some cases, capable of expressing the product of that polynucleotide. In addition to therapeutic considerations, cells expressing polynucleotides of the present invention may prove useful in the context of screening for agents that induce, repress, inhibit, augment, interfere with, block, abrogate, stimulate or enhance the function of Rad51, Xrcc3, or Rad51 /Xrcc3 complex.

A. Polynucleotides Encoding Rad51, Xrcc3, or Rad51/Xrcc3 Complex

Polynucleotides according to the present invention may encode an entire Rad51 or Xrcc3 gene, a domain, or any other fragment of the Rad51 or Xrcc3 sequences. The domain or fragment encoded by a polynucleotide of Rad51, Xrcc3, or Rad51/Xrcc3 complex may or may not retain DNA repair (or other) activity capable of forming a protective complex. Exemplary RAD51 sequences can be found in Genbank, for example, Genbank Accession No. AF034956 (Homo sapiens RAD51D); Genbank Accession No. U84138 (Homo sapiens RAD51B); Genbank Accession No. AF029670 (Homo sapiens Rad51C); Genbank Accession No. AF029669 (Homo sapiens Rad51C); Genbank Accession No. U43652 (Arabidopsis thaliana); Genbank Accession No. AF034955 (Mus musculus); Genbank Accession No. AF064516 (Tetrahymena thermophila RAD51); Genbank Accession No. AF017729 (Oryctolagus cuniculus). Exemplary XRCC3 sequences include Genbank Accession No. AF035586 and Genbank Accession No. AF037222. Each of these sequences is incorporated herein by reference, one of skill in the art is referred to the Genbank database (http://www.ncbi.nlm.nih.gov/Entrez) which contains various other RAD51 and related sequences.

The polynucleotide may be derived from genomic DNA, i.e., cloned directly from the genome of a particular organism. In preferred embodiments, however, the polynucleotide would comprise complementary DNA (cDNA). Also contemplated is a cDNA plus a natural intron or an intron derived from another gene; such engineered molecules are sometime referred to as “mini-genes.” At a minimum, these and other polynucleotides of the present invention may be used as molecular weight standards in, for example, gel electrophoresis.

The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

Naturally, the present invention also encompasses the use of polynucleotides that are complementary, or essentially complementary, to Rad51 and Xrcc3. Polynucleotide sequences that are “complementary” are those that are capable of base-pairing according to the standard Watson-Crick complementary rules. As used herein, the term “complementary sequences” means polynucleotide sequences that are substantially complementary, as may be assessed by the same nucleotide comparison set forth above, or as defined as being capable of hybridizing to Rad51 or Xrcc3 under relatively stringent conditions such as those described herein. Such sequences may encode the entire Rad51 or Xrcc3 protein, or functional or non-functional fragments thereof.

It also is contemplated that a given Rad51 or Xrcc3 from a given species may be represented by natural variants that have slightly different nucleic acid sequences but, nonetheless, encode the same protein. As used in this application, the term “a polynucleotide encoding” refers to a polynucleotide molecule that has been isolated free of total cellular nucleic acid. The term “functionally equivalent codon” is used herein to refer to codons that encode the same amino acid, such as the six codons for arginine or serine (Table 1, below), and also refers to codons that encode biologically equivalent amino acids, as discussed in the following pages.

TABLE 1

| Amino Acids | | | Codons |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gin | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

Allowing for the degeneracy of the genetic code, sequences that have at least about 50%, usually at least about 60%, more usually about 70%, most usually about 80%, preferably at least about 90% and most preferably about 95% of nucleotides that are identical to Rad51 or Xrcc3 are polynucleotides that may be used in the present invention. Polynucleotides that are essentially the same as Rad51 or Xrcc3 are also functionally defined as sequences that are capable of hybridizing to a polynucleotide segment containing the complement of Rad51 or Xrcc3 under standard conditions.

The polynucleotides of the present invention include those encoding biologically functional equivalent Rad51 or Xrcc3 proteins and peptides. Such sequences may arise as a consequence of codon redundancy and amino acid functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by humans may be introduced through the application of site-directed mutagenesis techniques or may be introduced randomly and screened later for the desired function, as described below.

B. Oligonucleotide Probes and Primers

In the present invention, an oligonucleotide probe or primer may be utilized which encodes a domain or fragment of Rad51, Xrcc3, or Rad51/Xrcc3 complex. A Rad51, Xrcc3, or Rad51/Xrcc3 complex probe or primer may be used in a number of molecular biology techniques well known to those skilled in the art including, but not limited to, Southern or Northern blot hybridization, in situ hybridization, or polymerase chain reaction (PCR). One of exploiting probes and primers of the present invention is in site-directed, or site-specific mutagenesis. Site-specific mutagenesis is a technique useful in the preparation of individual peptides, or biologically functional equivalent proteins or peptides, through specific mutagenesis of the underlying DNA. The technique further provides a ready ability to prepare and test sequence variants, incorporating one or more of the foregoing considerations, by introducing one or more nucleotide sequence changes into the DNA. Site-specific mutagenesis allows for the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

The technique typically employs a bacteriophage vector that exists in both a single-stranded and double-stranded form. Typical vectors useful in site-directed mutagenesis include vectors such as the M13 phage. These phage vectors are commercially available and their use is generally well known to those skilled in the art. Double-stranded plasmids are also routinely employed in site directed mutagenesis, which eliminates the step of transferring the gene of interest from a phage to a plasmid.

In general, site-directed mutagenesis is performed by first obtaining a single-stranded vector, or melting of two strands of a double-stranded vector which includes within its sequence a DNA sequence encoding the desired protein. An oligonucleotide primer bearing the desired mutated sequence is synthetically prepared. This primer is then annealed with the single-stranded DNA preparation, taking into account the degree of mismatch when selecting hybridization conditions, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected that include recombinant vectors bearing the mutated sequence arrangement.

The preparation of sequence variants of the selected gene using site-directed mutagenesis is provided as a means of producing potentially useful species and is not meant to be limiting, as there are other ways in which sequence variants of genes may be obtained. For example, recombinant vectors encoding the desired gene may be treated with mutagenic agents, such as hydroxylamine, to obtain sequence variants.

C. Antisense Constructs

If Rad51 and Xrcc3 interact as a complex to facilitate DNA repair as the inventors propose, a useful strategy to increase the susceptibility of certain cells, such as cancerous cells, to DNA damaging agents may be to prevent the formation of, or block the expression of, the Rad51/Xrcc3 complex. While Rad51 is essential for viability, Xrcc3 is not essential for the survival of eukaryotic cells. Antisense RNA treatments are one way of blocking the expression of Xrcc3 in a cell. Antisense technology also may be used to “knock-out” the function of Xrcc3 in the development of cell lines or transgenic mice for research, diagnostic and screening purposes, or therapeutic purposes. In addition, Rad51 function in a cell may also be reduced by antisense RNA treatments, potentially increasing the sensitivity of a cell to DNA damaging agents.

Antisense methodology takes advantage of the fact that nucleic acids tend to pair with “complementary” sequences. The term “complementary” is intended to refer to polynucleotides that are capable of base-pairing according to the standard Watson-Crick complementarity rules. That is, the larger purines will base pair with the smaller pyrimidines to form combinations of guanine paired with cytosine (G:C) and either adenine paired with thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense construct polynucleotides, when introduced into a target cell, specifically bind to their target polynucleotide and interfere with transcription, RNA processing, transport, translation and/or stability. Antisense RNA constructs, or DNA encoding such antisense RNAs, may be employed to inhibit gene transcription, translation, or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology are also contemplated. For example, an antisense construct which has limited regions of high homology, but also contains a non-homologous region (e.g., ribozyme; see below) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

D. Ribozymes

Another approach for addressing the “dominant negative” inactivation of Xrcc3 function, as well as partial inactivation of Rad51, is through the use of ribozymes. Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cook, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cook et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cook et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). Recently, it was reported that ribozymes elicited genetic changes in some cells lines to which they were applied; the altered genes included the oncogenes H-ras, c-fos and genes of HIV. Most of this work involved the modification of a target mRNA, based on a specific mutant codon that is cleaved by a specific ribozyme.

E. Vectors for Cloning, Gene Transfer and Expression

Within certain embodiments expression vectors are employed to express the Rad51, Xrcc3, or Rad51 /Xrcc3 complex polypeptide products, which can then be purified and, for example, used to vaccinate animals to generate antisera or monoclonal antibodies with which further studies may be conducted. In other embodiments, the expression vectors are used in therapeutic applications. Expression requires that appropriate signals be provided in the vectors, which include various regulatory elements, such as enhancers/promoters from both viral and mammalian sources that drive expression of the genes of interest in host cells. Elements designed to optimize messenger RNA stability and translatability in host cells are also defined. The conditions for the use of a number of dominant drug selection markers for establishing permanent, stable cell clones expressing the products are also provided, as is an element that links expression of the drug selection markers to expression of the polypeptide.

### (i) Regulatory Elements

Throughout this application, the term “expression construct” is meant to include any type of genetic construct containing a polynucleotide coding for a gene product in which part or all of the polynucleotide encoding sequence is capable of being transcribed. The transcript may be translated into a protein, but it need not be. In certain embodiments, expression includes both transcription of a gene and translation of mRNA into a gene product. In other embodiments, expression only includes transcription of the polynucleotide encoding a gene of interest.

In preferred embodiments, the polynucleotide encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the polynucleotide to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

The particular promoter employed to control the expression of a polynucleotide sequence of interest is not believed to be important, so long as it is capable of directing the expression of the polynucleotide in the targeted cell. Thus, where a human cell is targeted, it is preferable to position the polynucleotide coding region adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized.

Selection of a promoter that is regulated in response to specific physiologic or synthetic signals can permit inducible expression of the gene product. For example in the case where expression of a transgene, or transgenes when a multicistronic vector is utilized, is toxic to the cells in which the vector is produced in, it may be desirable to prohibit or reduce expression of one or more of the transgenes. Examples of transgenes that may be toxic to the producer cell line are pro-apoptotic and cytokine genes. Several inducible promoter systems are available for production of viral vectors where the transgene product may be toxic.

The ecdysone system (Invitrogen, Carlsbad, Calif.) is one such system. This system is designed to allow regulated expression of a gene of interest in mammalian cells. It consists of a tightly regulated expression mechanism that allows virtually no basal level expression of the transgene, but over 200-fold inducibility. The system is based on the heterodimeric ecdysone receptor of Drosophila, and when ecdysone or an analog such as muristerone A binds to the receptor, the receptor activates a promoter to turn on expression of the downstream transgene high levels of mRNA transcripts are attained. In this system, both monomers of the heterodimeric receptor are constitutively expressed from one vector, whereas the ecdysone-responsive promoter which drives expression of the gene of interest is on another plasmid. Engineering of this type of system into the gene transfer vector of interest would therefore be useful. Cotransfection of plasmids containing the gene of interest and the receptor monomers in the producer cell line would then allow for the production of the gene transfer vector without expression of a potentially toxic transgene. At the appropriate time, expression of the transgene could be activated with ecdysone or muristeron A.

Another inducible system that would be useful is the Tet-Off™ or Tet-On™ system (Clontech, Palo Alto, Calif.) originally developed by Gossen and Bujard (Gossen and Bujard, 1992; Gossen et al., 1995). This system also allows high levels of gene expression to be regulated in response to tetracycline or tetracycline derivatives such as doxycycline. In the Tet-On™ system, gene expression is turned on in the presence of doxycycline, whereas in the Tet-Off™ system, gene expression is turned on in the absence of doxycycline. These systems are based on two regulatory elements derived from the tetracycline resistance operon of E. coli. The tetracycline operator sequence to which the tetracycline repressor binds, and the tetracycline repressor protein. The gene of interest is cloned into a plasmid behind a promoter that has tetracycline-responsive elements present in it. A second plasmid contains a regulatory element called the tetracycline-controlled transactivator, which is composed, in the Tet-Off™ system, of the VP16 domain from the herpes simplex virus and the wild-type tertracycline repressor. Thus in the absence of doxycycline, transcription is constitutively on. In the Tet-On™ system, the tetracycline repressor is not wild type and in the presence of doxycycline activates transcription. For gene therapy vector production, the Tet-Off™ system would be preferable so that the producer cells could be grown in the presence of tetracycline or doxycycline and prevent expression of a potentially toxic transgene, but when the vector is introduced to the patient, the gene expression would be constitutively on.

An inducible system particularly useful with the present invention is a radiation-inducible system. Ionizing radiation-inducible promoters include a CArG domain of an Egr-1 promoter, a los promoter, a c-jun promoter, and a TNF-α promoter, which can be operatively linked to a protein expression region. In this regard, U.S. Pat. No. 5,612,318, dealing with induction of expression from these promoters, is specifically incorporated by reference.

In some circumstances, it may be desirable to regulate expression of a transgene in a gene therapy vector. For example, different viral promoters with varying strengths of activity may be utilized depending on the level of expression desired. In mammalian cells, the CMV immediate early promoter if often used to provide strong transcriptional activation. Modified versions of the CMV promoter that are less potent have also been used when reduced levels of expression of the transgene are desired. When expression of a transgene in hematopoetic cells is desired, retroviral promoters such as the LTRs from MLV or MMTV are often used. Other viral promoters that may be used depending on the desired effect include SV40, RSV LTR, HIV-1 and HIV-2 LTR, adenovirus promoters such as from the E1A, E2A, or MLP region, AAV LTR, cauliflower mosaic virus, HSV-TK, and avian sarcoma virus.

Similarly tissue specific promoters may be used to effect transcription in specific tissues or cells so as to reduce potential toxicity or undesirable effects to non-targeted tissues. For example, promoters such as the PSA, probasin, prostatic acid phosphatase or prostate-specific glandular kallikrein (hK2) may be used to target gene expression in the prostate.

In certain indications, it may be desirable to activate transcription at specific times after administration of the gene therapy vector. This may be done with such promoters as those that are hormone or cytokine regulatable. For example in gene therapy applications where the indication is a gonadal tissue where specific steroids are produced or routed to, use of androgen or estrogen regulated promoters may be advantageous. Such promoters that are hormone regulatable include MMTV, MT-1, ecdysone and RuBisco. Other hormone regulated promoters such as those responsive to thyroid, pituitary and adrenal hormones are expected to be useful in the present invention. Cytokine and inflammatory protein responsive promoters that could be used include K and T Kininogen (Kageyama et al., 1987), c-fos, TNF-alpha, C-reactive protein (Arcone et al., 1988), haptoglobin (Oliviero et al., 1987), serum amyloid A2, C/EBP alpha, IL-1, IL-6 (Poli and Cortese, 1989), Complement C3 (Wilson et al., 1990), IL-8, alpha-1 acid glycoprotein (Prowse and Baumann, 1988), alpha-1 antitypsin, lipoprotein lipase (Zechner et al., 1988), angiotensinogen (Ron et al., 1991), fibrinogen, c-jun (inducible by phorbol esters, TNF-alpha, UV radiation, retinoic acid, and hydrogen peroxide), collagenase (induced by phorbol esters and retinoic acid), metallothionein (heavy metal and glucocorticoid inducible), Stromelysin (inducible by phorbol ester, interleukin-1 and EGF), alpha-2 macroglobulin and alpha-1 antichymotrypsin.

Tumor specific promoters such as osteocalcin, hypoxia-responsive element (HRE), MAGE-4, CEA, alpha-fetoprotein, GRP78/BiP and tyrosinase may also be used to regulate gene expression in tumor cells. Other promoters that could be used according to the present invention include Lac-regulatable, chemotherapy inducible (e.g. MDR), and heat (hyperthermia) inducible promoters, radiation-inducible (e.g., EGR (Joki et al., 1995)), Alpha-inhibin, RNA pol III tRNA met and other amino acid promoters, U1 snRNA (Bartlett et al., 1996), MC-1, PGK, β-actin and α-globin. Many other promoters that may be useful are listed in Walther and Stein (1996).

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are frequently overlapping and contiguous, often seeming to have a very similar modular organization.

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read through from the cassette into other sequences.

### (ii) Selectable Markers

In certain embodiments of the invention, the cells contain polynucleotide constructs of the present invention, and a cell may be identified in vitro or in vivo by including a marker in the expression construct. Such markers will confer an identifiable change to the cell permitting easy identification of cells containing the expression construct. Usually the inclusion of a drug selection marker aids in cloning and in the selection of transformants, for example, genes that encode neomycin, puromycin, hygromycin, DHFR, GPT, HPRT, zeocin, and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the polynucleotide encoding a gene product. Further examples of selectable markers are well known to one of skill in the art.

### (iii) Multigene Constructs and IRES

In certain embodiments of the invention, internal ribosome binding sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picanovirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins, and selectable markers. In this way, expression of several proteins can be simultaneously engineered into a cell with a single construct and a single selectable marker.

### (iv) Delivery of Expression Vectors

There are a number of ways to introduce expression vectors into cells. In certain embodiments of the invention, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kb of foreign genetic material but can be readily introduced in a variety of cell lines and laboratory animals (Nicolas and Rubenstein, 1988; Temin, 1986).

One of the preferred methods for in vivo delivery involves the use of an adenovirus expression vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus. Knowledge of the genetic organization of adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNAs issued from this promoter possess a 5′-tripartite leader (TPL) sequence which makes them preferred mRNAs for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone and is the source of vector-borne cytotoxicity. Also, the replication deficiency of the E1-deleted virus is incomplete. For example, leakage of viral gene expression has been observed with the currently available vectors at high multiplicities of infection (MOI) (Mulligan, 1993).

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Recently, Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 hours. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 hours.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a polynucleotide encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

There are certain limitations to the use of retrovirus vectors in all aspects of the present invention. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This can result from recombination events in which the intact- sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Lentiviruses can also be used as vectors in the present application. In addition to the long-term expression of the transgene provided by all retroviral vectors, lentiviruses present the opportunity to transduce nondividing cells and potentially achieve regulated expression. The development of lentiviral vectors requires the design of transfer vectors to ferry the transgene with efficient encapsidation of the transgene RNA and with full expression capability, and of a packaging vector to provide packaging machinery in trans but without helper virus production. For both vectors, a knowledge of packaging signal is required-the signal to be included in the transfer vector but excluded from the packaging vector. Exemplary human lentiviruses are human immunodeficiency virus type 1 and type 2 (HIV-1 and HIV-2). HIV-2 is likely better suited for gene transfer than HIV-1 as it is less pathogenic and thus safer during design and production; its desirable nuclear import and undesirable cell-cycle arrest functions are segregated on two separate genes (Arya et al., 1998; Blomer et al., 1997).

AAV utilizes a linear, single-stranded DNA of about 4700 base pairs. Inverted terminal repeats flank the genome. Two genes are present within the genome, giving rise to a number of distinct gene products. The first, the cap gene, produces three different virion proteins (VP), designated VP-1, VP-2 and VP-3. The second, the rep gene, encodes four non-structural proteins (NS). One or more of these rep gene products is responsible for transactivating AAV transcription.

The three promoters in AAV are designated by their location, in map units, in the genome. These are, from left to right, p5, p19 and p40. Transcription gives rise to six transcripts, two initiated at each of three promoters, with one of each pair being spliced. The splice site, derived from map units 42-46, is the same for each transcript. The four non-structural proteins apparently are derived from the longer of the transcripts, and three virion proteins all arise from the smallest transcript.

AAV is not associated with any pathologic state in humans. Interestingly, for efficient replication, AAV requires “helping” functions from viruses such as herpes simplex virus I and II, cytomegalovirus, pseudorabies virus and, of course, adenovirus. The best characterized of the helpers is adenovirus, and many “early” functions for this virus have been shown to assist with AAV replication. Low level expression of AAV rep proteins is believed to hold AAV structural expression in check, and helper virus infection is thought to remove this block.

The terminal repeats of an AAV vector can be obtained by restriction endonuclease digestion of AAV or a plasmid such as psub201, which contains a modified AAV genome (Samulski et al. 1987), or by other methods known to the skilled artisan, including but not limited to chemical or enzymatic synthesis of the terminal repeats based upon the published sequence of AAV. The ordinarily skilled artisan can determine, by well-known methods such as deletion analysis, the minimum sequence or part of the AAV ITRs which is required to allow function, i.e. stable and site-specific integration. The ordinarily skilled artisan also can determine which minor modifications of the sequence can be tolerated while maintaining the ability of the terminal repeats to direct stable, site-specific integration.

AAV-based vectors have proven to be safe and effective vehicle for gene delivery in vitro, and these vectors are now being developed and tested in pre-clinical and clinical stages for a wide range of applications in potential gene therapy, both ex vivo and in vivo. However, wide variations in AAV transduction efficiency in different cells and tissues in vitro as well as in vivo has been repeatedly observed (Ponnazhagan et al., 1997b; 1997c; 1997d; 1997d) and others (Carter and Flotte, 1996; Chatterjee et al., 1995; Ferrari et al., 1996; Fisher et al., 1996; Flotte et al., 1993; Goodman et al., 1994; Kaplitt et al., 1994; 1996, Kessler et al., 1996; Koeberl et al., 1997; Mizukami et al., 1996; Xiao et al., 1996).

AAV-mediated efficient gene transfer and expression in the lung has led to clinical trials for the treatment of cystic fibrosis (Carter and Flotte, 1996; Flotte et al., 1993). Similarly, the prospects for treatment of muscular dystrophy by AAV-mediated gene delivery of the dystrophin gene to skeletal muscle, of Parkinson's disease by tyrosine hydroxylase gene delivery to the brain, of hemophilia B by Factor IX gene delivery to the liver, and potentially of myocardial infarction by vascular endothelial growth factor gene to the heart, appear promising since AAV-mediated transgene expression in these organs has recently been shown to be highly efficient (Fisher et al., 1996; Flotte et al., 1993; Kaplitt et al., 1994; 1996; Koeberl et al., 1997; McCown et al., 1996; Ping et al., 1996; Xiao et al., 1996).

Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. The hepatotropism and persistence (integration) were particularly attractive properties for liver-directed gene transfer. Chang et al. (1991) recently introduced the chloramphenicol acetyltransferase (CAT) gene into duck hepatitis B virus genome in the place of the polymerase, surface, and pre-surface coding sequences. It was co-transfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In order to effect expression of sense or antisense gene constructs, the expression construct must be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsulated in an infectious viral particle.

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the expression construct has been delivered into the cell the polynucleotide encoding the gene of interest may be positioned and expressed at different sites. In certain embodiments, the polynucleotide encoding the gene may be stably integrated into the genome of the cell. This integration may be in the cognate location and orientation via homologous recombination (gene replacement) or it may be integrated in a random, non-specific location (gene augmentation). In yet further embodiments, the polynucleotide may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the polynucleotide remains is dependent on the type of expression construct employed.

In yet another embodiment of the invention, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

In still another embodiment of the invention, transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa and hepatoma cells. Nicolau et al., (1987) accomplished successful liposome-mediated gene transfer in rats after intravenous injection.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. Since these expression constructs have been successfully employed in transfer and expression of polynucleotides in vitro and in vivo, then they are applicable for the present invention. Where a bacterial promoter is employed in the DNA construct, it also will be desirable to include within the liposome an appropriate bacterial polymerase.

Other expression constructs which can be employed to deliver a polynucleotide encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

In other embodiments, the delivery vehicle may comprise a ligand and a liposome. For example, Nicolau et al. (1987) employed lactosyl-ceramide, a galactose-terminal asialganglioside, incorporated into liposomes and observed an increase in the uptake of the insulin gene by hepatocytes. Thus, it is feasible that a polynucleotide encoding a particular gene also may be specifically delivered into a cell type such as lung, epithelial or tumor cells, by any number of receptor-ligand systems with or without liposomes. For example, epidermal growth factor (EGF) may be used as the receptor for mediated delivery of a polynucleotide encoding a gene in many tumor cells that exhibit upregulation of EGF receptor. Mannose can be used to target the mannose receptor on liver cells. Also, antibodies to CD5 (CLL), CD22 (lymphoma), CD25 (T-cell leukemia) and MAA (melanoma) can similarly be used as targeting moieties.

In certain embodiments, gene transfer may more easily be performed under ex vivo conditions. Ex vivo gene therapy refers to the isolation of cells from an animal, the delivery of a polynucleotide into the cells in vitro, and then the return of the modified cells back into an animal. This may involve the surgical removal of tissue/organs from an animal or the primary culture of cells and tissues.

### (v) Tissue Cell Culture

Primary mammalian cell cultures may be prepared in various ways. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented and are disclosed herein by reference (Freshner, 1992).

One embodiment of the foregoing involves the use of gene transfer to immortalize cells for the production of proteins. The gene for the protein of interest may be transferred as described above into appropriate host cells followed by culture of cells under the appropriate conditions. The gene for virtually any polypeptide may be employed in this manner. The generation of recombinant expression vectors, and the elements included therein, are discussed above. Alternatively, the protein to be produced may be an endogenous protein normally synthesized by the cell in question.

Examples of useful mammalian host cell lines are Vero and HeLa cells and cell lines of Chinese hamster ovary (CHO), W138, BHK, COS-7, 293, HepG2, NIH3T3, RIN and MDCK cells. In addition, a host cell strain may be chosen that modulates the expression of the inserted sequences, or modifies and process the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed.

A number of selection systems may be used including, but not limited to, HSV thymidine kinase, hypoxanthine-guanine phosphoribosyltransferase and adenine phosphoribosyltransferase genes, in tk-, hgprt- or aprt- cells, respectively. Also, anti-metabolite resistance can be used as the basis of selection for dhfr, that confers resistance to; gpt, that confers resistance to mycophenolic acid; neo, that confers resistance to the aminoglycoside G418; and hygro, that confers resistance to hygromycin.

Animal cells can be propagated in vitro in two modes: as non-anchorage dependent cells growing in suspension throughout the bulk of the culture or as anchorage-dependent cells requiring attachment to a solid substrate for their propagation (i.e., a monolayer type of cell growth).

Non-anchorage dependent or suspension cultures from continuous established cell lines are the most widely used means of large scale production of cells and cell products. However, suspension cultured cells have limitations, such as tumorigenic potential and lower protein production than adherent T-cells.

Large scale suspension culture of mammalian cells in stirred tanks is a common method for production of recombinant proteins. Two suspension culture reactor designs are in wide use—the stirred reactor and the airlift reactor. The stirred design has successfully been used on an 8000 liter capacity for the production of interferon. Cells are grown in a stainless steel tank with a height-to-diameter ratio of 1:1 to 3:1. The culture is usually mixed with one or more agitators, based on bladed disks or marine propeller patterns. Agitator systems offering less shear forces than blades have been described. Agitation may be driven either directly or indirectly by magnetically coupled drives. Indirect drives reduce the risk of microbial contamination through seals on stirrer shafts.

The airlift reactor, also initially described for microbial fermentation and later adapted for mammalian culture, relies on a gas stream to both mix and oxygenate the culture. The gas stream enters a riser section of the reactor and drives circulation. Gas disengages at the culture surface, causing denser liquid free of gas bubbles to travel downward in the downcorner section of the reactor. The main advantage of this design is the simplicity and lack of need for mechanical mixing. Typically, the height-to-diameter ratio is 10:1. The airlift reactor scales up relatively easily, has good mass transfer of gases and generates relatively low shear forces.

III. Rad51, Xrcc3, and Rad51/Xrcc3 Complex Polypeptides

According to the present invention, Rad51 and Xrcc3 may interact as a complex to facilitate DNA repair. While these molecules are structurally distinct, they are part of a group of functionally-related molecules involved in DNA repair. In addition to the complete Rad51, Xrcc3, and Rad51/Xrcc3 complex molecules, the present invention also relates to fragments of the polypeptides that may or may not retain the DNA repair (or other) activity. Fragments including the N-terminus of the molecule may be generated by genetic engineering of translation stop sites within the coding region. Alternatively, treatment of the Rad51, Xrcc3, and Rad51/Xrcc3 complex molecules with proteolytic enzymes, known as protease, can produces a variety of N-terminal, C-terminal and internal fragments. These fragments may be purified according to known methods, such as precipitation (e.g., ammonium sulfate), HPLC, ion exchange chromatography, affinity chromatography (including immunoaffinity chromatography) or various size separations (sedimentation, gel electrophoresis, gel filtration).

A. Fusion Proteins

A specialized kind of insertional variant is the fusion protein. This molecule generally has all or a substantial portion of the native molecule, linked at the N- or C-terminus, to all or a portion of a second polypeptide. For example, fusions typically employ leader sequences from other species to permit the recombinant expression of a protein in a heterologous host. Another useful fusion includes the addition of a immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions. One particular fusion of interest would include all or a portion of the native Rad51 molecule, linked at the N- or C-terminus, to all or a portion of a Xrcc3 polypeptide. Fusion to a polypeptide that can be used for purification of the substrate-Rad51/Xrcc3 complex would serve to isolated the substrate for identification and analysis.

B. Purification of Proteins

It may be desirable to purify the Rad51/Xrcc3 complex or variants thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography.

C. Synthetic Peptides

The present invention also describes smaller Rad51-related, Xrcc3-related, and Rad51/Xrcc3 complex-related peptides for use in various embodiments of the present invention. Because of their relatively small size, the peptides of the invention also can be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

IV. Methods for Screening Active Compounds

The present invention also contemplates the use of the Rad51/Xrcc3 complex or fragment thereof, and polynucleotides coding therefor, in the screening of compounds for activity in either stimulating Rad51/Xrcc3 complex formation, overcoming the lack of the Rad51/Xrcc3 complex, or blocking the effect of a mutant Rad51/Xrcc3 complex. These assays may make use of a variety of different formats and may depend on the kind of “activity” for which the screen is being conducted. Contemplated functional “read-outs” include binding to a compound, inhibition of binding to a substrate, ligand, receptor or other binding partner by a compound, phosphatase activity, anti-phosphatase activity, phosphorylation of Rad51/Xrcc3 complex, dephosphorylation of Rad51/Xrcc3 complex, inhibition or stimulation of cell-to-cell signaling, growth, metastasis, cell division, cell migration, soft agar colony formation, contact inhibition, invasiveness, angiogenesis, apoptosis, tumor progression or other malignant phenotype.

A. In Vitro Assays

In one embodiment, the invention will be applied to the screening of compounds that bind to the Rad51/Xrcc3 complex or fragments thereof. The polypeptides or fragments may be either free in solution, fixed to a support, or expressed in or on the surface of a cell. Either the polypeptides or the compound may be labeled, thereby permitting a determination of binding.

In another embodiment, the assay may measure the inhibition of binding of Rad51/Xrcc3 complex to a natural or artificial substrate or binding partner. Competitive binding assays can be performed in which one of the agents is labeled. Usually, the polypeptide will be the labeled species. One may measure the amount of free label versus bound label to determine binding or inhibition of binding.

Another technique for high throughput screening of compounds is described in WO 84/03564. Large numbers of small peptide test compounds are synthesized on a solid substrate, such as plastic pins or some other surface. The peptide test compounds are reacted with Rad51/Xrcc3 complex and washed. Bound polypeptide is detected by various methods.

Purified Rad51/Xrcc3 complex can be coated directly onto plates for use in the aforementioned drug screening techniques. However, non-neutralizing antibodies to the polypeptides can be used to immobilize the polypeptides to a solid phase. Also, fusion proteins containing a reactive region (preferably a terminal region) may be used to link the Rad51/Xrcc3 complex active region to a solid phase.

Various cell lines containing wild-type or natural or engineered mutations in Rad51/Xrcc3 complex can be used to study various functional attributes of the Rad51/Xrcc3 complex and how a candidate compound affects these attributes. Methods for engineering mutations are described elsewhere in this document. In such assays, the compound would be formulated appropriately, given its biochemical nature, and contacted with a target cell. Depending on the assay, culture may be required. The cell may then be examined using a number of different physiologic assays. Alternatively, molecular analysis may be performed in which the function of Rad51/Xrcc3 complex, or related pathways, may be explored. This may involve assays such as those for protein expression, enzyme function, substrate utilization, phosphorylation states of various molecules including Rad51/Xrcc3 complex, cAMP levels, mRNA expression (including differential display of whole cell or polyA RNA) and others.

In yet another embodiment, proteins that interact with the Rad51/Xrcc3 complex may be identified by using a yeast two-hybrid system or a co-immunoprecipitation assay. The yeast two-hybrid system may be used to identify new protein targets for pharmaceutical intervention, determine the specific residues involved in a given protein-protein interaction, and find compounds that modulate protein interactions. The yeast two-hybrid system can also be used to identify previously unknown proteins that interact with a target protein by screening a two-hybrid library. The yeast two-hybrid system is outlined in U.S. Pat. No. 5,283,173 (incorporated herein by reference), and is a technique well know to those of skill in the art. Briefly, the method is designed to detect an interaction between a first test protein and a second test protein, in vivo, using reconstitution of the activity of a transcriptional activator. Two chimeric proteins that express hybrid proteins are prepared. The first hybrid protein contains the DNA-binding domain of a transcriptional activator fused to the first test protein, while the second hybrid protein contains a transcriptional activation domain fused to the second test protein. If the two test proteins interact, the two domains of the transcriptional activator are brought into close proximity, resulting in the transcription of a marker gene that contains a binding site for the DNA-binding domain. An assay can be performed to detect activity of the marker gene.

All yeast two-hybrid systems share a set of common elements: 1) a plasmid that directs the synthesis of a “bait”; the bait is a known protein which is fused to a DNA binding domain, 2) one or more reporter genes (“reporters”) with upstream DNA binding sites for the bait, and 3) a plasmid that directs the synthesis of proteins fused to activation domains and other useful moieties (“activation tagged proteins” or “prey” ). All current systems direct the synthesis of proteins that carry the activation domain at the amino terminus of the fusion, facilitating the expression of open reading frames encoded by cDNAs. DNA binding domains used in the yeast two-hybrid systems include the native E. coli LexA repressor protein (Gyuris et al., 1993), and the GAL4 protein (Chien et al., 1991). Some reporter genes that may be utilized in the yeast system included HIS3, LEU2, and lacZ.

Although most two-hybrid systems use yeast, mammalian variants may also be utilized. In one system, interaction of activation tagged VP16 derivatives with a Gal4-derived bait drives expression of reporters that direct the synthesis of Hygromycin B phosphotransferase, Chloramphenicol acetyltransferase, or CD4 cell surface antigen (Fearon et al., 1992). In another system, interaction of VP16-tagged derivatives with Gal4-derived baits drives the synthesis of SV40 T antigen, which in turn promotes the replication of the prey plasmid, because the plasmid carries a SV40 origin (Vasavada et al., 1991).

Protein-protein interactions may also be studied by using biochemical techniques such as cross-linking, co-immunoprecipitation, and co-fractionation by chromatography, which are well known to those skilled in the art. The co-immunoprecipitation technique consists of (i) generating a cell lysate; (ii) adding an antibody to the cell lysate; (iii) precipitating and washing the antigen; and (iv) eluting and analyzing the bound proteins (Phizicky and Fields, 1995). The antigen used to generate the antibody can be a purified protein, or a synthetic peptide coupled to a carrier. Both monoclonal and polyclonal antibodies can be utilized in co-immunoprecipitation, or alternatively, a protein can be used which carries an epitope tag recognized by a commercially available antibody.

B. In Vivo Assays

Treatment of animals with Rad51, Xrcc3, Rad51/Xrcc3 complex, or test compounds will involve the administration of the compound, in an appropriate form, to the animal. Administration will be by any route the could be utilized for clinical or non-clinical purposes, including but not limited to oral, nasal, buccal, rectal, vaginal or topical. Alternatively, administration may be by intratracheal instillation, bronchial instillation, intradermal, subcutaneous, intramuscular, intraperitoneal or intravenous injection. Specifically contemplated are systemic intravenous injection, regional administration via blood or lymph supply and intratumoral injection.

Determining the effectiveness of a compound in vivo may involve a variety of different criteria. Such criteria include, but are not limited to, survival, reduction of tumor burden or mass, arrest or slowing of tumor progression, elimination of tumors, inhibition or prevention of metastasis, increased activity level, improvement in immune effector function and improved food intake.

C Rational Drug Design

The goal of rational drug design is to produce structural analogs of biologically active polypeptides or compounds with which they interact (agonists, antagonists, inhibitors, binding partners, etc.). By creating such analogs, it is possible to fashion drugs which are more active or stable than the natural molecules, which have different susceptibility to alteration, or which may affect the function of various other molecules. In one approach, one would generate a three-dimensional structure for Rad51/Xrcc3 complex or a fragment thereof. This could be accomplished by x-ray crystallograph, computer modeling or by a combination of both approaches. An alternative approach, “alanine scan,” involves the random replacement of residues throughout molecule with alanine, with the resulting effect on the function of the molecule determined.

It also is possible to isolate a Rad51/Xrcc3 complex specific antibody, selected by a functional assay, and then solve its crystal structure. In principle, this approach yields a pharmacore upon which subsequent drug design can be based. It is possible to bypass protein crystallograph altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

Thus, one may design drugs which have improved Rad51/Xrcc3 complex activity or which act as stimulators, inhibitors, agonists, antagonists of Rad51/Xrcc3 complex, or molecules affected by Rad51/Xrcc3 complex function. By virtue of recombinant DNA technology, sufficient amounts of Rad51/Xrcc3 complex can be produced to perform crystallographic studies. In addition, knowledge of the polypeptide sequences permits computer employed predictions of structure-function relationships.

V. Methods for Treating Cellular DNA Damage

The present invention also involves, in another embodiment, the prevention or treatment of cellular damage in normal cells in response to DNA damaging agents. The types of cellular damage that may be treated, according to the present invention, is limited only by the involvement of Rad51 or Xrcc3. By involvement, it is not a requirement that either Rad51 or Xrcc3 be mutated or abnormal. Since DNA damaging agents are often used to treat cancer, killing healthy cells as well as cancerous cells, it is contemplated that Rad51, Xrcc3, or Rad51/Xrcc3 complex therapy may be used to increase the cellular resistance of healthy cells in response to DNA damage.

A. Genetic Based Therapies

One of the therapeutic embodiments contemplated by the present invention is the intervention, at the molecular level, in events involved in cellular DNA repair. Specifically, the present inventors intend to provide, to a cell or an animal patient, an expression construct capable of producing Rad51, Xrcc3, or Rad51/Xrcc3 complex. Expression of these molecules will provide healthy cells with increased resistance to DNA damaging agents. The lengthy discussion of polynucleotides employed herein is incorporated into this section by reference. Particularly preferred polynucleotides are contained in viral vectors such as adenovirus, adeno-associated virus, herpesvirus, vaccinia virus and retrovirus.

Those of skill in the art are well aware of how to apply gene delivery to in vivo and ex vivo situations. For viral vectors, one generally will prepare a viral vector stock. Depending on the kind of virus and the titer attainable, one will deliver 1×10⁴, 1×10⁵, 1×10⁶, 1×10⁷, 1×10⁸, 1×10⁹, 1×10¹⁰, 1×10¹¹ or 1×10¹² infectious particles to the patient. Similar figures may be extrapolated for other non-viral formulations by comparing relative uptake efficiencies.

In a different embodiment, ex vivo gene therapy is contemplated. This approach is particularly suited, although not limited, to treatment of bone marrow associated cancers. In an ex vivo embodiment, cells from the patient are removed and maintained outside the body for at least some period of time. During this period, a therapy is delivered, after which the cells are reintroduced into the patient; hopefully, any tumor cells in the sample have been killed.

Autologous bone marrow transplant (ABMT) is an example of ex vivo gene therapy. Basically, the notion behind ABMT is that the patient will serve as his or her own bone marrow donor. Thus, a normally lethal dose of irradiation or chemotherapeutic may be delivered to the patient to kill tumor cells, and the bone marrow repopulated with the patients own cells that have been maintained (and perhaps expanded) ex vivo. Because, bone marrow often is contaminated with tumor cells, it is desirable to purge the bone marrow of these cells. Use of gene therapy to accomplish this goal is yet another way Rad51, Xrcc3, or Rad51/Xrcc3 complex may be utilized according to the present invention.

B. DNA Damaging Agents

Agents or factors suitable for use in a combined therapy with Rad51, Xrcc3, or Rad51/Xrcc3 complex are any chemical compound or treatment method that induces DNA damage when applied to a cell. Such agents and factors include radiation and waves that induce DNA damage such as, γ-irradiation, X-rays, UV-irradiation, microwaves, electronic emissions, and the like. A variety of chemical compounds, also described as “chemotherapeutic agents,” function to induce DNA damage, all of which are intended to be of use in the combined treatment methods disclosed herein. Chemotherapeutic agents contemplated to be of use, include, e.g., adriamycin, 5-fluorouracil (5FU), etoposide (VP-16), camptothecin, actinomycin-D, mitomycin C, cisplatin (CDDP) and even hydrogen peroxide. The invention also encompasses the use of a combination of one or more DNA damaging agents, whether radiation-based or actual compounds, such as the use of X-rays with cisplatin or the use of cisplatin with etoposide. In certain embodiments, the use of cisplatin in combination with a Rad51, Xrcc3, or Rad51/Xrcc3 complex polynucleotide is particularly preferred.

Classical chemotherapeutic agents include steroids, antimetabolites, anthracycline, vinca alkaloids, antibiotics, alkylating agents, epipodophyllotoxin and anti-tumor agents such as neocarzinostatin (NCS), adriamycin and dideoxycytidine; mammalian cell cytotoxins, such as interferon-α (IFN-α), interferon-βγ (IFN-βγ), interleukin-12 (IL-12) and tumor necrosis factor-α (TNF-α); plant-, fungus- and bacteria-derived toxins, such as ribosome inactivating protein, gelonin, α-sarcin, aspergillin, restrictocin, ribonucleases, diphtheria toxin, Pseudomonas exotoxin, bacterial endotoxins, the lipid A moiety of a bacterial endotoxin, ricin A chain, deglycosylated ricin A chain and recombinant ricin A chain; as well as radioisotopes.

Cisplatin has been widely used to treat cancer, with efficacious doses used in clinical applications of 20 mg/m² for 5 days every three weeks for a total of three courses. Cisplatin is not absorbed orally and must therefore be delivered via injection intravenously, subcutaneously, intratumorally or intraperitoneally.

Agents that damage DNA also include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 mg/M² at 21 day intervals for adriamycin, to 35-50 mg/m² for etoposide intravenously or double the intravenous dose orally.

Agents that disrupt the synthesis and fidelity of nucleic acid precursors and subunits also lead to DNA damage. As such a number of nucleic acid precursors have been developed. Particularly useful are agents that have undergone extensive testing and are readily available. As such, agents such as 5-fluorouracil (5-FU), are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used.

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage DNA, on the precursors of DNA, the replication and repair of DNA, and the assembly and maintenance of chromosomes. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The inventors propose that the delivery of Rad51, Xrcc3, or Rad51/Xrcc3 complex polynucleotides to patients will be an efficient method for delivering an effective therapy to counteract the DNA damaging treatments often employed to treat cancer. Similarly, the chemotherapy or radiotherapy may be directed to a particular, affected region of the subjects body.

C. Formulations and Routes for Administration to Patients

Where clinical applications are contemplated, it will be necessary to prepare pharmaceutical compositions - expression vectors, virus stocks, proteins, antibodies and drugs—in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions, described supra.

The active compounds also may be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

VI. Methods for Enhancing Cellular DNA Damage in Cancer Cells

The present invention includes, in another embodiment, the treatment of cancer through the functional inactivation of the Rad51/Xrcc3 complex. The types of cancer that may be treated are limited only by the involvement of Rad51 or Xrcc3. By involvement, it is not a requirement that either Rad51 or Xrcc3 be mutated or abnormal. Since the formation of a Rad51/Xrcc3 complex appears to regulate DNA damage repair, it is contemplated that preventing the formation of the complex in a cell using an antisense RNA strategy will enhance the susceptibility of a cell to DNA damaging agents. A single-chain antibody strategy may also be used to block the function of the Rad51/Xrcc3 complex in a cell. A wide variety of cancers may be treated using such a strategy, including cancers of the brain (glioblastoma, astrocytoma, oligodendroglioma, ependymomas), lung, liver, spleen, kidney, lymph node, pancreas, small intestine, blood cells, colon, stomach, breast, endometrium, prostate, testicle, ovary, skin, head and neck, esophagus, bone marrow, blood or other tissue. The invention may also be used in benign neoplasms, including meningiomas, arteriovenous malformations, and hemangiomas.

In many contexts, it is not necessary that the tumor cell be killed or induced to undergo normal cell death or “apoptosis.” Rather, to accomplish a meaningful treatment, all that is required is that the tumor growth be slowed to some degree. It may be either that the tumor growth is completely blocked, or that some tumor regression is achieved. Clinical terminology such as “remission” and “reduction of tumor” burden also are contemplated given their normal usage.

A. Antisense RNA

One therapeutic embodiment contemplated by the present invention is the intervention, at the molecular level, in the events involved in cellular DNA repair. In particular, the present inventors intend to prevent the formation of the Rad51/Xrcc3 complex in cancer cells within an animal patient. The efficacy of radiotherapy and chemotherapy will be improved if a strategy can be used to specifically increase the susceptibility of cancer cells to DNA damaging agents in a patient. Formation of the Rad51/Xrcc3 complex can be blocked by expressing Rad51 antisense RNA, Xrcc3 antisense RNA, or both antisense RNAs in cancer cells. The inventors propose that the absence of the Rad51/Xrcc3 complex will enhance the susceptibility of cancer cells to DNA damaging agents because the Rad51/Xrcc3 complex will not be present to aid in DNA repair. The lengthy discussion of polynucleotides employed herein is incorporated into this section by reference. Particularly preferred polynucleotides are contained in viral vectors such as adenovirus, adeno-associated virus, herpesvirus, vaccinia virus and retrovirus.

Various routes are contemplated for delivery of Rad51 antisense RNA, Xrcc3 antisense RNA, or both antisense RNAs to cancer cells in an animal patient. The discussion on routes for administration to patients employed herein is incorporated into this section by reference. Systemic delivery in animal patients is contemplated by the invention. If a discrete tumor mass is identified in a patient, a variety of direct, local and regional approaches may be taken.

B. Combined Therapy with Traditional Chemotherapy or Radiotherapy

Tumor cell resistance to DNA damaging agents represents a major problem in clinical oncology. One goal of current cancer research is to find ways to improve the efficacy of chemotherapy and radiotherapy. One way is by combining such traditional therapies with gene therapy. For example, the herpes simplex-thymidine kinase (HS-tk) gene, when delivered to brain tumors by a retroviral vector system, successfully induced susceptibility to the antiviral agent ganciclovir (Culver et al., 1992). In the context of the present invention, it is contemplated that Rad51 antisense RNA, Xrcc3 antisense RNA, or both antisense RNAs could similarly be used in conjunction with chemotherapeutic or radiotherapeutic intervention.

To kill cells, inhibit cell growth, inhibit metastasis, inhibit angiogenesis or otherwise reverse or reduce the malignant phenotype of tumor cells using the methods and compositions of the present invention, one would generally contact a “target” cell with Rad51 antisense RNA, Xrcc3 antisense RNA, or both antisense RNA expression constructs and at least one other DNA damaging agent. These compositions would be provided in a combined amount effective to kill or inhibit proliferation of the cell. This process may involve contacting the cells with the antisense RNA expression constructs and the agent(s) or factor(s) at the same time. This may be achieved by contacting the cell with a single composition or pharmacological formulation that includes both agents, or by contacting the cell with two distinct compositions or formulations, at the same time, wherein one composition includes the expression constructs and the other includes the agent.

Alternatively, the Rad51 antisense RNA, Xrcc3 antisense RNA, or both antisense RNA treatments may precede or follow the other DNA damaging agent treatment by intervals ranging from minutes to weeks. In embodiments where the other agent and antisense RNA expression constructs are applied separately to the cell, one would generally ensure that a significant period of time did not expire between the time of each delivery, such that the agent and expression constructs would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It is also conceivable that more than one administration of either Rad51 antisense RNA, Xrcc3 antisense RNA, both antisense RNAs, or the other agent will be desired. Various combinations may be employed, where Rad51 antisense RNA, Xrcc3 antisense RNA, or both antisense RNAs are “A” and the other DNA damaging agent is “B”, as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated. Again, to achieve cell death, both agents are delivered to a cell in a combined amount effective to kill the cell.

Agents or factors suitable for use in a combined therapy are any chemical compound or treatment method that induces DNA damage when applied to a cell. Such agents and factors include radiation and waves that induce DNA damage, such as γ-irradiation, X-rays, UV-irradiation, microwaves, electronic emissions, and the like. A variety of chemical compounds, also described as “chemotherapeutic agents,” function to induce DNA damage, all of which are intended to be of use in the combined treatment methods disclosed herein. Chemotherapeutic agents contemplated to be of use, include, e.g., adriamycin, 5-fluorouracil (5FU), etoposide (VP-16), camptothecin, actinomycin-D, mitomycin C, cisplatin (CDDP) and even hydrogen peroxide. The invention also encompasses the use of a combination of one or more DNA damaging agents, whether radiation-based or actual compounds, such as the use of X-rays with cisplatin or the use of cisplatin with etoposide. In certain embodiments, the use of cisplatin in combination with Rad51 antisense RNA, Xrcc3 antisense RNA, or both antisense RNA expression constructs is particularly preferred as this compound.

In treating cancer according to the invention, one would contact the tumor cells with a DNA damaging agent in addition to antisense RNA expression constructs. This may be achieved by irradiating the localized tumor site with radiation such as X-rays, UV-light, γ-rays or even microwaves. Alternatively, the tumor cells may be contacted with the agent by administering to the subject a therapeutically effective amount of a pharmaceutical composition comprising a compound such as adriamycin, 5-fluorouracil, etoposide, camptothecin, actinomycin-D, mitomycin C, or more preferably, cisplatin. The agent may be prepared and used as a combined therapeutic composition, or kit, by combining it with Rad51 antisense RNA, Xrcc3 antisense RNA, or both antisense RNA expression constructs, as described above.

Agents that directly cross-link nucleic acids, specifically DNA, are envisaged to facilitate DNA damage leading to a synergistic, antineoplastic combination with the antisense RNA strategy. Agents such as cisplatin, and other DNA alkylating agents may be used. The lengthy discussion of DNA damaging agents employed herein is incorporated into this section by reference.

The inventors propose that the regional delivery of Rad51 antisense RNA, Xrcc3 antisense RNA, or both antisense RNA expression constructs to patients with cancer will be a very efficient method for counteracting the clinical disease. Similarly, the chemotherapy or radiotherapy may be directed to a particular, affected region of the subjects body. Alternatively, systemic delivery of antisense RNA expression constructs and/or the DNA damaging agent may be appropriate in certain circumstances, for example, where extensive metastasis has occurred.

C. Single-Chain Antibodies

Another therapeutic embodiment of the present invention contemplates the use of single-chain antibodies to block the activity of the Rad51/Xrcc3 complex in cells, in particular cancer cells. Single-chain antibodies can be synthesized by a cell, targeted to particular cellular compartments, and used to interfere in a highly specific manner with cell growth and metabolism (Richardson and Marasco, 1995). Recently, single-chain antibodies were utilized for the phenotypic knockout of growth-factor receptors, the functional inactivation of p21ras, and the inhibition of HIV-1 replication. Intracellular antibodies offer a simple and effective alternative to other forms of gene inactivation, as well as demonstrate a clear potential as reagents for cancer therapy and for the control of infectious diseases. Single-chain antigen-binding proteins also represent potentially unique molecules for targeted delivery of drugs, toxins, or radionuclides to a tumor site, and show increased accessibility to tumor cells in vivo (Yokoda et al., 1992). Single-chain antibodies that bind Rad51, Xrcc3, or Rad51/Xrcc3 complex can be introduced into a cell to functionally inactivate the Rad51/Xrcc3 complex.

Methods for the production of single-chain antibodies are well known to those of skill in the art. The skilled artisan is referred to U.S. Pat. No. 5,359,046, (incorporated herein by reference) for such methods. A single-chain antibody is created by fusing together the variable domains of the heavy and light chains using a short peptide linker, thereby reconstituting an antigen binding site on a single molecule.

Single-chain antibody variable fragments (scFvs) in which the C-terminus of one variable domain is tethered to the N-terminus of the other via a 15 to 25 amino acid peptide or linker, have been developed without significantly disrupting antigen binding or specificity of the binding (Bedzyk et al., 1990; Chaudhary et al., 1990). These Fvs lack the constant regions (Fc) present in the heavy and light chains of the native antibody.

It is also contemplated by the present invention that single-chain antibody therapy can be combined with chemotherapeutic or radiotherapeutic intervention. The discussion of combined therapy with traditional chemotherapy or radiotherapy employed herein is incorporated into this section by reference.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Materials and Methods

Cell lines: CHO cell lines AA8, irs1SF, PXR3 and CXR3 (Thompson et al., 1980; Tebbs et al., 1995) were used by the inventors in a series of experiments, as well as xrs5 and K1 (Jeggo and Kemp, 1983). A mouse scid cell line (designated UC-RCO-1) originated from fibroblasts grown from the skin of a C3H scid mouse; mouse 4102 cells (a fibrosarcoma cell line from C3H mice) was used as a control. The CHO cell lines AA8, irs1SF, PXR3 and CXR3 were grown using DME/F-12 (3:1), 10% FBS, 100 units/ml penicillin, and 100 μg/ml streptomycin. The CHO cell lines K1 and xrs5 were grown in McCoy's medium, 10% FBS and pen/strep. Mouse cells were grown in either DME/F-12 (3:1), 5% FBS, and pen/strep or MEM, 5% FBS, and pen/strep.

Irradiation Studies: Exponentially growing cultures in 60 cm dishes were X-irradiated with a Maxitron generator (General Electric) operating at 250 kV and 26 mA with a dose rate of 114 cGy/min. Dishes were returned to the incubator immediately after treatment. For dose response studies, cells were incubated for 3 hours after irradiation before being harvested by trypsin treatment and fixed with 1% paraformaldehyde. For time course analysis, cells were irradiated with 900 cGy and then incubated for various periods of time before being harvested and fixed.

Cisplatin treatment: For dose response studies, the cultures were washed twice in serum-free medium and then incubated for 1 hour in serum-free medium containing varying concentrations of cisplatin (Bristol Laboratories). After the incubation, the dishes were washed three times in serum-free medium and complete medium was added. Cultures were then placed at 37° C. for 3 hours at which time a single-cell suspension was obtained with trypsin/EDTA and the cells were prepared for immunostaining. For time course studies, cell cultures were washed twice in serum-free medium and incubated for one hour in serum-free medium containing 10 μM cisplatin. After incubation, the cells were washed and incubated in complete medium. At the appropriate times the cells were harvested by trypsin treatment and prepared for immunostaining.

Immunostaining: Fixed cells were transferred to slides using a Cytospin 2 (Shandon, Pittsburgh, Pa.) centrifuge. Slides were washed twice in Tris-buffered saline (TBS) and incubated for 15 minutes in TBS 1% bovine serum albumin BSA at room temperature. The wash was removed and anti-Rad51 antibody in TBS 1% BSA was applied. The slides were incubated overnight at 4° C. in a humidified chamber. The slides were washed twice in TBS and secondary antibody (fluorescein-conjugated goat anti-rabbit; Molecular Probes, Inc., Eugene, Oreg.) was applied at a 1:1000 dilution in the dark. The slides were incubated at room temperature in the dark for 1 hour. The slides were washed twice with TBS and stained with DAPI (0.1 μg/ml in TBS) for 5 minutes. The slides were dried and coverslips mounted with Vectashield mounting medium (Vector Laboratories Inc., Burlingame, Calif.).

For most studies affinity purified a-HsRad51 antibody (Terasawa et al., 1995) was used. In some studies, a-HsRad51 serum was used and gave essentially identical results to those obtained with the affinity purified IgG, to give faint, non-specific background staining. This faint background was distinguished from the bright, Rad51 -specific signal by competition with purified Rad51 protein; addition of the pure protein eliminated the bright radiation-inducible foci, but not the faint background staining pattern.

Microscopy: Immunostained samples were examined via standard epifluorescence microscopy using a Ziess Photoscope III through a 100X objective. Digital images were obtained with a CCD camera (Imagepoint, Photometrics Inc., Tucson, Ariz.). A single focal plane was documented for each nucleus. Therefore, the numbers presented are less than the total number of foci per nucleus. Samples consisted of focus counts for 50 unselected nuclei. The Kruskal-Wallis test was used to determine the statistical significance of observed differences between samples. Color images that combine fluorescein and DAPI staining patterns were generated by converting grayscale images to pseudo color and then merging the patterns electronically using I.P. Lab Spectrum software (Signal Analytics Corp., Vienna, Va.).

Western analysis: Samples were prepared for Western analysis (Terasawa et al., 1995) using Immobilon membranes (Millipore Corp., Bedford, Mass.). Non-specific binding was prevented by pre-treatment of filters with a blocking solution which was TBS containing 5% dry milk and 0.1% TWEEN 20. The primary antibody was a-HsRad51 IgG at 0.5 μg/ml and the secondary antibody (goat anti-rabbit peroxidase conjugate, Boehringer Mannheim Corp., Indianapolis, Ind.) at a 1/5000 fold dilution. Signals were detected by chemiluminescence (Renaissance, Dupont/NEN, Boston, Mass.). The level of signal detected was shown to be in the linear range of detection by parallel analysis of a dilution series of protein extract from AA8 cells. Measurement of cross-reactivity of a-Rad51 for Xrcc3p by Western analysis was performed using pure Rad51p and Xrcc3p. Xrcc3p was expressed in bacterial cells (BL21/pLysS) bearing the plasmid pET29-55 (obtained from Dr. Larry Thompson, Lawrence Livermore National Laboratory) and purified on a Ni²⁺-column (Clontech; Palo Alto, Calif.).

Clonogenic survival assays: To measure the radiation resistance of various cell lines, between 100 and 40,000 cells were plated in 100 mm cell culture dishes. Four hours after plating, cells were irradiated as described above and immediately returned to the incubator. 10-12 days later the colonies were fixed and stained with crystal violet. Only colonies of >50 cells were scored as survivors.

Expression of HsRAD51 in CHO cells: Constructs containing the HsRAD51 gene (pEG915) bearing an N-terminal His6-epitope tag were transiently co-transfected together with a plasmid bearing an MHC cell-surface antigen (pH-2-L^{d}) into CHO cell-lines PXR3 and irs1SF cells. Transient transfections were performed using TransIT™ reagents (Panvera Corp., Madison, Wis.) according to the manufacturers' recommendations. Cells were also co-transfected with the vector lacking the RAD51 gene (pEBVHisB, Invitrogen), or with no DNA (in addition to the pH-2-L^{d} plasmid), as controls. The response of all transfectants to X-irradiation damage was analyzed, in three independent studies, by indirect immunostaining with a-His6 antibody at a 1:200 dilution (to detect for the plasmid-expressed Rad51p) and mouse anti-H-2L^{d} monoclonal antibody; secondary antibodies were conjugated with Texas Red and FITC (Southern Biotech, Birmingham, Ala.), respectively. The inventors then searched cytologically for the presence or absence of subnuclear foci. Expression of His6-tagged HsRad51p (41.3 kD fusion protein vs. 37 kD HsRad51) in transfectants was confirmed by Western blot analysis with a-His6 antibody and the a-hsRad51 antibody.

### EXAMPLE 2

### Effects of Radiation on Cell Lines

Radiation induces the formation of subnuclear foci in CHO cells: Cycling cells of CHO line AA8 were exposed to X-rays and then incubated for various periods of time. Following incubation, the cells were fixed and indirectly immunostained with a-HsRAD51 antibodies. The cell lines were treated with 9 Gy of X-rays 3 hours prior to fixation. Examination of stained samples by fluorescence microscopy revealed that the antibody was localized to multiple subnuclear foci. For simplicity, the subnuclear structures detected in CHO cells by a-HsRAD51 staining are referred to as Rad51 foci. In addition to radiation-induced foci, a small number of foci (typically fewer than 5) were detected in cells that were either untreated or fixed immediately after radiation treatment. These results are qualitatively similar to previous observations in human fibroblasts and lymphocytes (Haaf et al., 1995; Tashiro et al., 1996; Tashiro et al., 1996), although the number of foci detected in untreated cells is somewhat less than that reported previously.

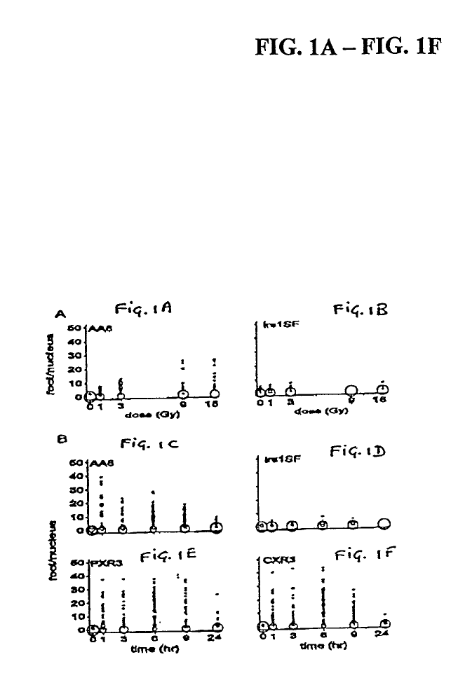

The maximum response, both in terms of the fraction of cells that showed a response and in terms of the number of Rad51 foci present per responding cell, was obtained with 9 Gy of X-irradiation (FIG. 1A and FIG. 1B). The properties of Rad51 focus induction are most easily described by using a threshold of 5 foci/cell to distinguish cells that show a focus formation response from those that do not. Using this threshold, the fraction of responding cells reached a maximum 6 hours after irradiation. The average number of foci per responding cell reached a maximum 1 to 3 hours following irradiation and typically remained at this induced level until after 6 hours (FIG. 1C-FIG. 1D). There was substantial heterogeneity in the number of foci detected per nucleus even under conditions that gave maximum numbers of foci. For example, 40% of AA8 cells contained 5 or fewer foci 6 hours after a dose of 9 Gy (FIG. 1C-FIG. 1D). Preliminary results suggest that the cells that fail to produce Rad51 foci are those in the G1 phase of the cell cycle at the time of treatment. A low Rad51 expression level may be responsible for the failure of G1 CHO cells to produce foci given that a low Rad51 expression level has been reported for human fibroblasts in G1 (Yamamoto et al., 1996).

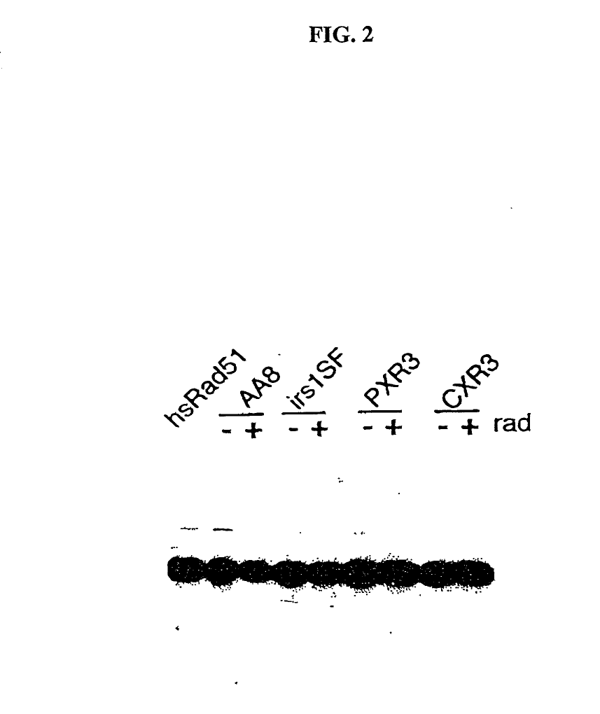

Radiation promotes redistribution of Rad51 protein: The dramatic change in the pattern of a-Rad51 staining caused by radiation is not associated with a corresponding change in Rad51 steady state protein levels. Western analysis was carried out to determine the steady state level of Rad51 protein before and after irradiation (FIG. 2). Little or no difference in steady state protein levels was observed when treated and untreated samples were compared, suggesting that radiation-induced focus formation is not an indirect consequence of increased protein levels.

Radiation-induced Rad51 redistribution is defective in the radiation-sensitive cell line irs1SF, but is rescued by expressing XRCC3 cDNA: The CHO line designated irs1SF is a radiation sensitive derivative of AA8 (Fuller and Painter, 1988). Radiation did not induce Rad51 focus formation in irs1SF cells (FIG. 1); the number of foci detected in irs1SF following radiation was never significantly different from the number detected in untreated AA8 or irs1SF cells (p=0.4). Western analysis of steady state Rad51 levels showed the level of Rad51 in irs1SF cells was essentially the same as in AA8, both before and after radiation, indicating that the failure of irs1SF cells to form foci did not result from the failure of these cells to express Rad51 protein (FIG. 2). These results indicate that irs1SF is defective in damage induced Rad51 foci.

Two independent derivatives of irs1SF, designated PXR3 and CXR3, have been described that display near normal radiation resistance (Tebbs et al., 1995). These derivatives were obtained by transfection of irs1SF with XRCC3 cDNA expression plasmids. PXR3 and CXR3 cells form Rad51 foci in response to X-rays. The numbers of Rad51 foci detected in these two radiation-resistant derivatives were essentially identical to the numbers detected in parental AA8 (FIG. 1A-FIG. 1F). Western analysis did not reveal any effect of the XRCC3 construct on steady state Rad51 protein levels (FIG. 2). Since Xrcc3 is distantly related to Rad51 (22% identical at the predicted amino acid level), the inventors considered the possibility that the foci detected are assemblies of Xrcc3 protein rather than Rad51 protein. However, Western analysis of the two proteins overexpressed in E. coli demonstrated a 10⁴-fold lower reactivity of a-Rad51 specific antibody to Xrcc3p as compared to HsRad51. Moreover, Western blot analysis allows the resolution of Rad51p and Xrcc3, yet no signal was ever detected at the position of Xrcc3, providing further evidence that the signal detected cytologically is not Xrcc3. Thus, it is highly unlikely that the signal observed in cytological preparations results from cross reaction of the antibody to Xrcc3. Western analysis also indicates that if the probe cross reacts with another protein, this protein must have an electrophoretic mobility identical to HsRad51 (note that highly purified HsRad51 is used as a standard on the Western blot shown). Together these results provide strong evidence that Xrcc3 promotes the subnuclear assembly of hamster Rad51 following radiation.

Xrcc3 also promotes the formation of human Rad51 subnuclear foci in CHO cells. As a further demonstration that Xrcc3 specifically influences nuclear distribution of Rad51 following radiation, the inventors performed transient co-transfection studies on PXR3 and irs1SF cells. Cells were cotransfected with a plasmid bearing a construct that expresses a his6-tagged derivative of human Rad51 protein, pEG915 (Haaf et al., 1995). This construct was cotransfected with a plasmid expressing a murine MHC cell-surface antigen marker, H-2-L^{d} (pH-2L^{d}), to identify transfected cells. As a control, cells were also cotransfected with vector alone (in addition to pH-2-L^{d}). The inventors observed subnuclear foci only in Rad51-his6 transfected PXR3 cells that had been irradiated prior to examination. Parallel control studies indicated that appearance of foci depended on the Rad51-his6 construct and on irradiation. Most importantly, no focus containing cells were observed after transfection of irs1SF with Rad51-his6 under the same conditions used for detection of focus-positive PXR3 cells. This observation provides a second line of evidence indicating that Xrcc3 can promote subnuclear assembly of Rad51 following radiation.

In these studies the staining foci detected by the anti-His6 probe were similar in terms of the number of foci/positive staining cell and in terms of the staining intensity of foci compared to those detected with anti-Rad51. However, the number of nuclei containing foci recognized by the a-his6 antibody was only about 1 in 170 transfected cells. The low frequency of focus containing cells may have been a consequence of the heterologous expression system. Western analysis using a-Rad51 antibodies (not anti-his6 antibodies) indicated that although the steady state level of the his6-tagged protein was the same in PXR3 and irs1SF cells, this level was quite low compared to that of the endogenous hamster protein, suggesting that only a small fraction of transfected cells express the protein encoded by the construct.

### EXAMPLE 3

### The Crosslinking Agent Cisplatin Induces XRCC3-dependent Rad51 foci

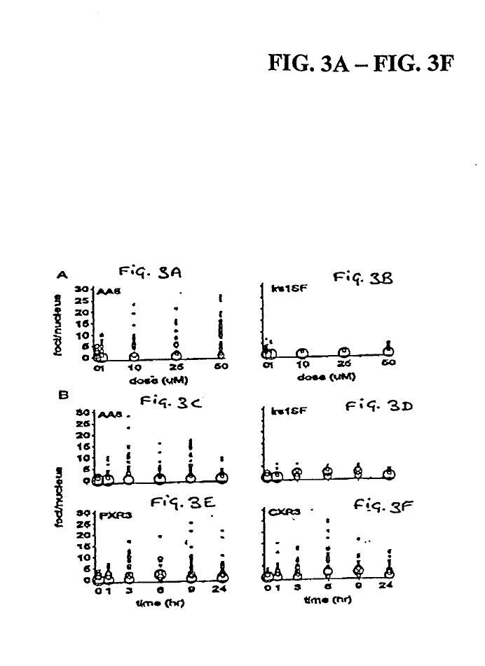

Xrcc3 promotes resistance to crosslinking agents as evidenced by the ability of cDNA constructs that encode XRCC3 to rescue the cisplatin sensitivity of irs1SF cells (Tebbs et al., 1995). To implicate Rad51 in the response to cisplatin and to show that Xrcc3 promotes Rad51 focus formation in response to a second type of DNA damage, CHO lines were treated with various amounts of cisplatin and then stained with a-Rad51 antibodies (FIG. 3A-FIG. 3F). Cisplatin treatment induced up to 30 Rad51 foci in AA8 FIG. 3C), PXR3 (FIG. 3E) and CXR3 (FIG. 3F) cells. In contrast, no induction of foci was detected following treatment of irs1SF cells with cisplatin. Cisplatin-induced Rad51 foci in AA8 cells tended to be fainter than foci induced by X-rays. Significant induction of foci was seen after treatment with 1 μM cisplatin for 1 hour, a dose that allows 50-80% of cells to survive (Tebbs et al., 1995). Near maximum induction of foci was seen at 10 μM.

### EXAMPLE 4

### DNA-PK Defective Cell Lines are Proficient in Formation of Rad51 foci

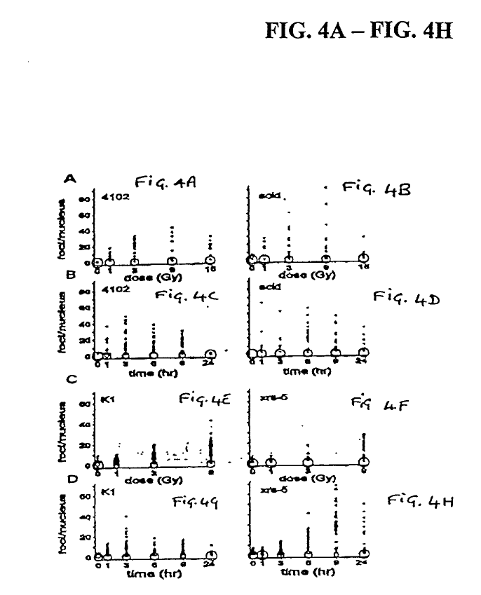

To determine if DNA-PK is required for Rad51 focus formation, a fibroblast line derived from a scid mouse was examined. The murine fibroblast line designated 4102, a fibrosarcoma line, was used as a control (FIG. 4A). While the two cell lines displayed the expected difference in radioresistance in clonogenic survival assays, the number of Rad51 foci detected following radiation treatment was not significantly different (p=0.58, FIG. 4A-FIG. 4D). The inventors also examined the CHO cell line xrs5, which has a DNA repair defect complemented by XRCC5, a human gene encoding the Ku86 subunit of DNA-PK (Boubnov et al., 1995; Smider et al., 1994). As with the scid cell line, the xrs5 line showed significant induction of Rad51 foci in response to X-rays (p=0.0001). Unexpectedly, the number of foci detected in xrs5 cells at 9 hours after a dose of 9 Gy was significantly greater than the number detected in the radiation resistant parent line (p=0.0001, FIG. 4G-FIG. 4H). Additional work will be required to determine if this difference between K1 and xrs5 depends on Ku86. In summary, induction of Rad51 foci was observed in two different DNA-PK defective cell lines indicating that focus formation does not depend on fully functional DNA-PK.

### EXAMPLE 5

### Defects in XRCC3 Renders Tumors Sensative to Cis-platinum

The sensitivity of XRCC3 defective cells to cis-platinum is also reflected in the ability of the drug to cause regression and cure of tumors in a mouse xenograft tumor model. Groups of 12 nude mice were injected in the hind limb with 5X105 XRCC3+ cells or xrcc3− cells. XRCC3+ lines (essentially equivalent to the PXR3 lines described above) and isogenic xrcc3− lines (equivalent to the irs1SF line described above), were injected into nude mice. Tumors were allowed to grow for 2 weeks (volumes of 300-500 mm3). On days 1-5 tumors were injected with 0.1 ml 100 μg/ml cisplatin in PBS or with the same volume of PBS alone. This dose was used because preliminary dose-response experiments showed it had a slight, but significant effect on the rate of increase of XRCC3+ tumors. Tumor volumes were measured at 3-5 day intervals starting on the first day of treatment. Fractional tumor volumes were calculated as the volume of the tumor on a particular day divided by the volume of the tumor on the day of the first treatment. The fractional tumor volumes in mice injected with XRCC3+ positive cells increased regardless of whether they received cisplatin treatment or the control treatment; the final fractional tumor volume for both the treated and untreated groups was about 5 on average. In contrast XRCC3+ tumors, the xrcc3− tumors showed a dramatic response to cisplatin treatment. Fractional tumor volume was about 5 on average for the untreated cells and about 0.05 on average for the tumors treated with cisplatin. Statistical analysis indicated that this difference in average fractional tumor volume was highly significant. These results demonstrate that the absence of xrcc3− function enhanced the ability of cisplatin to cause tumor regression and that inhibition of XRCC3 function can enhance the effectiveness of cisplatin as an anti-tumor agent.

Discussion

In summary, cells of the radiation-sensitive CHO line irs1SF fail to form Rad51 foci in response to radiation. A construct that encodes the XRCC3 gene, a relative of RAD51, restores both radiation resistance (Tebbs et al., 1995) and Rad51 focus formation to irs1SF. These results support the view that Rad51 foci reflect the mechanism that promotes cellular resistance to radiation. Given that Rad51, like RecA, functions in vitro by assembling into multimeric nucleoprotein filaments, it is likely that the damage-induced foci are Rad51 multimers assembled at damaged sites. If Rad51 foci represent productive repair intermediates they might be expected to reach a maximum as the repair capacity of the cell is saturated. This may be the case for cisplatin induced damage; 10 μM cisplatin is a dose that nearly saturates the repair capacity of the cell and gives near maximum levels of foci (Tebbs et al., 1995). On the other hand, efficient induction of Rad51 foci by X-rays requires doses that cause at least a 50-fold loss of viability (Tebbs et al., 1995). It is therefore possible that the repair capacity of the cell must be saturated for X-ray-induced complexes of Rad51 protein to be readily detected by the inventors' method. It should be noted, however, that there may be differences in the composition of Rad51-associated protein complexes that are induced by cisplatin and X-irradiation damage which may affect the appearance of Rad51 foci. The results of the inventors indicate that Xrcc3 promotes Rad51 focus formation in response to DNA damage. It is anticipated that Xrcc3 also promotes formation of the S-phase foci seen in lymphocytes and fibroblasts.

Rad51 foci form as a consequence of treatment with cisplatin, a DNA crosslinking agent that is widely used in chemotherapy. While XRCC3 has been shown to rescue cellular sensitivity to cisplatin, demonstration of a similar function for Rad51 is difficult because the RAD51 gene is essential for viability. However, a role for mammalian Rad51 protein in promoting resistance to cisplatin is expected because such a role has been demonstrated for yeast Rad51 (Jachymczyk et al., 1981). The Rad51 focus assay provides a means of detecting the involvement of mammalian Rad51 in DNA damage responses and has been used to show mammalian Rad51 is involved in the response to ionizing radiation, UV irradiation, and the alkylating agent MMS (Haaf, 1995). The inventors' studies show that cisplatin induces XRCC3-dependent Rad51 foci, indicating that mammalian Rad51 also plays a role in the response to this clinically important drug.

The failure of Rad51 to form damage-induced foci in irs1SF cells, and the rescue of this defect by XRCC3, suggest that participation of Rad51 in the normal response to DNA damage requires XRCC3. A mechanism through which XRCC3 promotes Rad51 function is suggested by recent “two-hybrid” studies showing that HsRad51 protein and Xrcc3 protein interact with one another directly. Although Rad51 may interact with Xrcc3, the inventors results presented here indicate that the Xrcc3-Rad51 interaction is biologically relevant, with the Xrcc3 protein promoting the assembly of, or stabilization of, a higher order Rad51-containing structure required for DNA repair. A related situation applies in yeast where ScRad51 focus formation depends on the RAD52, RAD55, and RAD57 genes (Gasior et al., 1998). These results, together with evidence for corresponding protein-protein interactions (Shinohara et al., 1992; Milne and Weaver, 1993; Hays et al., 1995; Johnson and Symington, 1995; Shen et al., 1996) suggest that, in both yeast and mammalian cells, Rad51 must interact directly with other repair proteins to assemble during DNA repair.

The combination of cytology and genetics used here provides a general method for characterizing the pathway of assembly of DNA repair proteins in vivo. Of particular interest for future studies of mammalian cells are the proteins encoded by the breast cancer genes BRCA1 and BRCA2, whose products were recently shown to interact directly with Rad51 (Scully et al., 1997; Sharan et al., 1997).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

Aboussekhra et al., Mol. Cell. Biol., 12:3224-3234, 1992.

Albala et al., Genomics, 46:476-479, 1997

Anderson and Lees-Miller, Crit. Rev. Eukaryo. Gene Express., 2:283-314, 1992.

Arcone, et al., Nucl. Acids Res., 16(8): 3195-3207, 1988.

Arya et al., Hum Gene Ther. 9(9): 1371-1380, 1998

Ashley et al., Chromosoma, 104:19-28, 1995.

Baichwal and Sugden, In: Gene Transfer, Kucherlapati R, ed., New York, Plenum Press, pp. 117-148, 1986.

Barany and Merrifield, The Peptides, Gross and Meienhofer, eds., Academic Press, New York, pp. 1-284, 1979.

Bartlett et al., Proc. Nat'l Acad. Sci. USA, 93:8852-8857, 1996.

Basile et al., Mol. Cell. Biol., 12:3235-3246, 1992.

Baumann, Benson, West, Cell, 87:757-766, 1996.

Bedzyk et al., J. Biol. Chem.,265:18615, 1990

Benvenisty and Neshif, Proc. Nat'l Acad. Sci USA, 83:9551-9555, 1986.

Bishop et al., Cell, 69:439-456, 1992.

Bishop, Cell, 79:1081-1092, 1994.

Blomer et al., J Virol. 71(9): 6641-6649, 1997

Blunt et al., Proc. Nat'l Acad. Sci. USA, 93:10285-10290, 1996.

Boubnov et al., Proc. Nat'l Acad. Sci USA, 92:890-894, 1995.

Byers et al., Cancer Res. 49(21): 6153-6160, 1989

Capaldi et al., Biochem. Biophys. Res. Comm., 76:425, 1977

Carter and Flotte, Curr. Top. Microbiol. Immunol., 218:119-144, 1996.

Chang et al., Hepatology, 14:124A, 1991.

Chatterjee, et al., Ann. N.Y. Acad. Sci., 770:79-90, 1995.

Chaudhary et al., Proc. Nat'l Acad. Sci., 87:9491, 1990

Chen and Okayama, Mol. Cell Biol., 7:2745-2752, 1987.

Chien et al., Proc. Nat'l Acad. Sci. USA, 88:9578-82, 1991.

Coffin, In: Virology, Fields et al., eds., Raven Press, New York, pp. 1437-1500, 1990.

Cook et al., Cell, 27:487496, 1981.

Couch et al., Am. Rev. Resp. Dis., 88:394-403, 1963.

Coupar et al., Gene, 68:1-10, 1988.

Cox and Lehman, Proc. Natl. Acad. Sci. USA, 78:6018-6022, 1981.

Culver et al., Science, 256:1550-1552, 1992.

Dosanjh et al., Nuc. Acids Res., 26:1179-1184, 1998.

Dubensky et al., Proc. Nat'l Acad. Sci. USA, 81:7529-7533, 1984.

Epenetos et al., Cancer Res. 46(6): 3183-3191, 1986

Errami et al., Mol. Cell. Biol., 16:1519-1526, 1996.

Fearon et al., Proc. Nat'l Acad Sci. USA, 89:7958-62, 1992.

Fechheimer et al., Proc. Nat'l Acad. Sci. USA, 84:8463-8467, 1987.

Ferkol et al., FASEB J., 7:1081-1091, 1993.

Ferrari, et al., J. Virol., 70:3227-3234, 1996.

Fisher et al., J. Virol., 70:520-532, 1996.

Flotte et al., Proc. Nat'l. Acad. Sci. USA, 90:10613-10617, 1993.

Forster and Symons, Cell, 49:211-220, 1987.

Fraley et al., Proc. Nat'l Acad. Sci. USA, 76:3348-3352, 1979.

Freshner, Animal Cell Culture: A Practical Approach, 2nd ed., Oxford/New York, IRL Press, Oxford University Press, 1992.

Friedberg et al., “DNA repair and mutagenesis,” A. S. M. Press, Washington, D.C., 1995.

Friedmann, Science, 244:1275-1281, 1989.

Fuller and Painter, Mutation Res., 193:109-121, 1988.

Gasior et al., Genes Dev., 12: 2208-2221, 1998.

Gerlach et al., Nature (London), 328:802-805, 1987.

Ghosh-Choudhury et al., EMBO J., 6:1733-1739, 1987.

Ghosh and Bachhawat, In: Liver diseases, targeted diagnosis and therapy using specific receptors and ligands, (Wu G, Wu C ed.), New York: Marcel Dekker, pp. 87-104, 1991.

Gomez-Foix et al, J. Biol. Chem., 267:25129-25134, 1992.

Goodman, et al., Blood, 84:1492-1500, 1994.

Gopal, Mol. Cell Biol., 5:1188-1190, 1985.

Gossen and Bujard, Proc. Nat'l Acad. Sci. USA, 89:5547-5551, 1992.

Gossen et al., Science, 268:1766-1769, 1995.

Gottlieb and Jackson, Cell, 72:131-142, 1993.

Graham & Prevec, Biotechnology, 20:363-390, 1992.

Graham and Prevec, In: Methods in Molecular Biology: Gene Transfer and Expression Protocols 7, E. J. Murray (ed.), Clifton, N.J., Humana Press, pp. 205-225. 1991.

Graham and Van Der Eb, Virology, 52:456-467, 1973.

Graham et al., J. Gen. Virol., 36:59-72, 1977.

Grunhaus and Horwitz, Seminar in Virology, 3:237-252, 1992.

Gupta et al., Proc. Nat'l Acad. Sci. USA, 94:463-468, 1997.

Gyuris et al., Cell, 75:791-803, 1993.

Haaf et al., Proc. Nat'l Acad. Sci. USA, 92:2298-2302, 1995.

Habu et al., Nucl. Acids Res., 24:470-477, 1996.

Harland and Weintraub, J. Cell Biol., 101:1094-1099, 1985.

Hays et al., Proc. Nat'l Acad. Sci. USA, 92:6925-6929, 1995.

Hermonat and Muzycska, Proc. Nat'l Acad. Sci. USA, 81:6466-6470, 1984.

Hersdorffer et al., DNA Cell Biol., 9:713-723, 1990.

Herz and Gerard, Proc. Nat'l Acad. Sci. USA, 90:2812-2816, 1993.

Horwich et al., J. Virol., 64:642-650, 1990.

Jachymczyk et al., Mol. Gen. Genet., 182:196-205, 1981.

Jeggo and Kemp, Mutat. Res., 112:313-327, 1983.

Johnson and Symington, Mol. Cell. Biol., 15:4843-4850, 1995.

Joki, et al., Human Gene Ther., 6:1507-1513, 1995.

Jones & Shenk, Cell, 13:181-188, 1978.

Joyce, Nature, 338:217-244, 1989.

Kageyama, et al., J. Biol. Chem., 262(5):2345-2351, 1987.

Kaneda et al., Science, 243:375-378, 1989.

Kaplitt et al., Methods, 10(3): 343-350, 1996

Kaplitt et al., Nat. Genet., 8:148-153, 1994.

Karlsson et al, EMBO J., 5:2377-2385, 1986.

Kato et al., J. Biol. Chem., 266:3361-3364, 1991.

Kessler et al., J. Leukocyte Biol., 60:729-736, 1996.

Kim and Cook, Proc. Nat'l Acad. Sci. USA, 84:8788-8792, 1987.

Kirchgessner et al., Science, 267:1178-1183, 1995.

Klein et al., Nature, 327:70-73, 1987.

Koeberl et al., Proc. Nat'l. Acad. Sci. USA, 94:1426-1431, 1997.

Kowalczykowski et al., Microbiol. Rev., 58:401-465, 1994.

Krasin and Hutchinson, J. Mol. Biol., 116:81-98, 1997.

Le Gal La Salle et al., Science, 259:988-990, 1993.

Levrero et al., Gene, 101: 195-202, 1991.

Li et al., Proc. Nat'l Acad. Sci USA, 93:10222-10227, 1996.

Lim and Hasty, Mol. Cell. Biol., 16:7133-7143, 1996.

Liu et al., Am. J. Hum. Genet., 57(A):147, 1995.

Lovett, Gene, 142:-103-106, 1994.

Macejak and Sarnow, Nature, 353:90-94, 1991.

Mann et al., Cell, 33:153-159, 1983.

Markowitz et al., J. Virol., 62:1120-1124, 1988.

McCown et al., Brain Res., 713:99-107, 1996.

Merrifield, Science, 232:341-347, 1986.

Michel and Westhof, J. Mol. Biol., 216:585-610, 1990.

Milne and Weaver, Genes Dev., 7:1755-1765, 1993.

Mizukami, et al., Virology, 217:124-130, 1996.

Mulligan, Science, 260:926-932, 1993.

Myers, EPO 0273085

Nelms et al., Science, 280:590-592, 1998.

Nicolas and Rubenstein, In: Vectors: A survey of molecular cloning vectors and their uses, Rodriguez and Denhardt (eds.), Stoneham: Butterworth, pp. 493-513, 1988.

Nicolau and Sene, Biochim. Biophys. Acta, 721:185-190, 1982.

Nicolau et al., Methods Enzymol., 149:157-176, 1987.

Olivierio, et al., EMBO J., 6(7):1905-1912, 1987.

Paskind et al., Virology, 67:242-248, 1975.

Pelletier and Sonenberg, Nature, 334:320-325, 1988.

Perales et al., Proc. Nat'l Acad. Sci. 91:4086-4090, 1994.

Phizicky and Fields, Microbiol. Rev., 59(1):94-123, 1995.

Ping, et al., Microcirculation, 3:225-228, 1996.

Poli and Cortese, Proc. Natl. Acad. Sci. USA, 86:8202-8206, 1989.

Ponnazhagan, et al., Gene, 190:203-210, 1997c.

Ponnazhagan, et al., Hum. Gene Ther., 8:275-284, 1997a.

Ponnazhagan, et al., J. Virol., 71:,1997b.

Ponnazhagan, et al., J. Virol., 71:3098-3104, 1997d.

Potter et al., Proc. Nat'l Acad. Sci. USA, 81:7161-7165, 1984.

Prowse and Baumann, Mol Cell Biol, 8(1):42-51, 1988.

Racher et al., Biotechnology Techniques, 9:169-174, 1995.

Ragot et al., Nature, 361:647-650, 1993.

Reinhold-Hurek and Shub, Nature, 357:173-176, 1992.

Renan, Radiother. Oncol., 19:197-218, 1990.

Rich et al., Hum. Gene Ther., 4:461-476, 1993.

Richardson and Marasco, Trends Biotechnol., 13(8):306-10, 1995.

Ridgeway, In: Vectors: A survey of molecular cloning vectors and their uses, Rodriguez R L, Denhardt D T, ed., Stoneham: Butterworth, pp. 467492, 1988.

Rippe et al., Mol. Cell Biol., 10:689-695, 1990.

Roca and Cox, Crit. Rev. in Biochem. and Mol. Biol., 25:415-456, 1991.

Ron, et al., Mol. Cell. Biol., 2887-2895, 1991.

Rosenfeld et al., Cell, 68:143-155, 1992.

Rosenfeld et al., Science, 252:431-434, 1991.

Ross et al., Cancer Res., 55:1235-1238, 1995.

Roux et al., Proc. Nat'l Acad. Sci. USA, 86:9079-9083, 1989.

Samulski et al., J. Virol., 6110:3096-3101, 1987.

Sands et al., Cancer Res. 48(1): 188-193, 1988.

Sarver et al., Science, 247:1222-1225, 1990.

Sato et al., DNA Res., 2:147-150, 1995.

Scanlon et al., Proc Nat'l Acad Sci USA, 88:10591-10595, 1991.

Scully et al., Cell, 88:265-275, 1997.

Sharan et al., Nature, 386:804-810, 1997.

Shen et al., J. Biol. Chem., 271:148-152, 1996.

Shinohara and Ogawa, T.I.B.S., 20:387-391, 1995.

Shinohara et al., Cell, 69:457-470, 1992.

Shinohara et al., Ogawa, Nature Genet., 4:239-243, 1993.

Smider et al., Science, 266:288-291, 1994.

Stahl, Genetics, 138:241-246, 1994.

Stassen et al., Curr. Genet, 31:144-157, 1997.

Stewart and Young, In: Solid Phase Peptide Synthesis, 2d. ed., Pierce Chemical Co., 1984.

Stratford-Perricaudet and Perricaudet, In: Human Gene Transfer, O. Cohen-Haguenauer et al., eds., John Libbey Eurotext, France, pp. 51-61, 1991.

Stratford-Perricaudet et al., Hum. Gene. Ther., 1:241-256, 1990.

Sung, Science, 265:1241-1243, 1994.

Taccioli et al., Science, 265:1442-1445, 1994.

Tam et al., J. Am. Chem. Soc., 105:6442, 1983.

Tashiro et al., Proc. Nat'l Acad. Sci. USA, 92:6354-6358, 1995.

Temin, In: Gene Transfer, Kucherlapati (ed.), New York: Plenum Press, pp. 149-188, 1986.

Terasawa et al., Ogawa, Genes Dev., 9:925-934, 1995.

Thompson et al., Environ. Mol. Mutaten., 27:68, 1996.

Thompson et al., Mutat. Res., 74:21-36, 1980.

Top et al., J. Infect. Dis., 124:155-160, 1971.

Tsuzuki et al., Proc. Nat'l Acad. Sci USA, 93, 6236-6240, 1996.

Tur-Kaspaet al., Mol. Cell Biol., 6:716-718, 1986.

U.S. Pat. No. 5,359,046,

U.S. Pat. No. 5,283,173

Varmus et al., Cell, 25:23-36, 1981.

Vasavada et al., Proc. Nat'l Acad. Sci. USA, 88:10686-90, 1991.

Wagner et al., Proc. Nat'l Acad. Sci. 87(9):3410-3414, 1990.

Walther and Stein, J. Mol. Med, 74:379-392, 1996.

Weiner et al., Cancer Res. 49(14): 4062-40671989

Wilson, et al., Mol. Cell. Biol., 6181-6191, 1990.

Wong et al., Gene, 10:87-94, 1980.

Wu and Wu, Adv. Drug Delivery Rev., 12:159-167, 1993.

Wu and Wu, Biochemistry, 27:887-892, 1988.

Wu and Wu, J. Biol. Chem., 262:4429-4432, 1987.

Xiao, et al., J. Virol., 70:8098-8108, 1996.

Yamamoto et al., Mo. Gen. Genet. 251, 1-12, 1996.

Yang et al., Proc. Nat'l Acad. Sci USA, 87:9568-9572, 1990.

Yokoda et al., Cancer Res. 52, 3402-3408, 1992.

Zechner et al., Mol. Cell. Biol., 2394-2401, 1988.

Zelenin et al., FEBS Lett., 280:94-96, 1991.

What is claimed is:

1. A method of producing a functional Rad51/Xrcc3 complex comprising:
(i) providing to a cell
(a) a first polynucleotide encoding a Rad51 polypeptide;
(b) a second polynucleotide encoding a Xrcc3 polypeptide; and
(ii) expressing said complex in a cell,
wherein the coexpression of said polypeptides allows for the formation of a functional Rad51/Xrcc3 complex.

2. The method of claim 1, wherein said first and said second polynucleotides are provided to said cell as naked DNA.

3. The method of claim 1, wherein said first and said second polynucleotides are provided to said cell through liposomal delivery or viral delivery.

4. The method of claim 1, wherein said first and said second polynucleotides are contained in different expression constructs and are under the control of a first and a second promoter, respectively.

5. The method of claim 4, wherein expression of said first and said second polynucleotides is controlled by a radiation-inducible promoter.

6. The method of claim 4, wherein said polynucleotides have a polyadenylation signal positioned 3′ to said first and said second polynucleotides, respectively.

7. The method of claim 4, wherein said expression constructs contain a selectable marker.

8. The method of claim 4, wherein said expression constructs are viral vectors.

9. The method of claim 4, wherein said first and said second polynucleotides are contained in the same expression construct and are under the control of a first and a second promoter, respectively.

10. The method of claim 9, wherein expression of said first and said second polynucleotides are controlled by a radiation-inducible promoter.

11. The method of claim 9, wherein said expression construct contains a first polyadenylation signal positioned 3′ to said first polynucleotide and a second polyadenylation signal positioned 3′ to said second polynucleotide.

12. The method of claim 9, wherein said expression construct contains a selectable marker.

13. The method of claim 9, wherein said expression construct is a viral vector.

14. The method of claim 9, wherein said first and said second polynucleotides are both under the control of a first promoter.

15. The method of claim 14, further comprising a polyadenylation signal positioned 3′ to said second polynucleotide.

16. The method of claim 14, wherein said first and said second polynucleotides are expressed as a fusion protein.

17. A method of inhibiting the formation of a functional Rad51/Xrcc3 complex comprising:
(i) providing to a cell a polynucleotide encoding a Xrcc3 antisense RNA; and
(ii) expressing said Xrcc3 antisense RNA in a cell,
wherein expression of said Xrcc3 antisense RNA blocks the expression of endogenous Xrcc3, thereby preventing the formation of a functional Rad51/Xrcc3 complex.

18. The method of claim 17, wherein said polynucleotide is provided to said cell as naked DNA.

19. The method of claim 17, wherein said polynucleotide is provided to said cell through liposomal delivery or viral delivery.

20. The method of claim 17, wherein said polynucleotide is contained in an expression construct under the control of a promoter.

21. The method of claim 20, wherein expression of said polynucleotide is controlled by a radiation-inducible promoter.

22. The method of claim 20, wherein said expression construct has a polyadenylation signal positioned 3′ to the polynucleotide.

23. The method of claim 20, wherein said expression construct contains a selectable marker.

24. The method of claim 20, wherein said expression construct is a viral vector.

25. The method of claim 17, wherein said Xrcc3 antisense RNA blocks the expression of said endogenous Xrcc3 by binding to the promoter, exon sequences, intron sequences, exon-intron splice junctions, or transcription start site of the Xrcc3 gene.

26. The method of claim 17, wherein said Xrcc3 antisense RNA blocks the expression of said endogenous Xrcc3 by binding to the translation start site or ribosomal binding site of Xrcc3 mRNA.

27. A method for identifying a candidate substance that modulates Rad51/Xrcc3 complex formation comprising:
(i) providing a Rad51 and a Xrcc3 under conditions suitable for Rad51/Xrcc3 complex formation;
(ii) contacting the components of step (a) with said candidate substance; and
(iii) determining the effect of said candidate substance on Rad51/Xrcc3 complex formation,
wherein an increase or decrease in Rad51/Xrcc3 complex formation, as compared to Rad51/Xrcc3 complex formation in the absence of said candidate substance, identifies said candidate substance as a modulator of Rad51/Xrcc3 complex formation.

28. The method of claim 27, wherein said conditions suitable for Rad51/Xrcc3 complex formation is exposure to DNA damage.

29. The method of claim 28, wherein said DNA damage is caused by ionizing radiation, ultraviolet radiation, cisplatin, adriamycin, 5-fluorouracil, etoposide, camptothecin, actinomycin-D, mitomycin C, verapamil or the DNA alkylating agent methylmethane sulfonate (MMS).

30. The method of claim 27, wherein said candidate substance is identified by utilizing said Rad51/Xrcc3 complex in a yeast two-hybrid system or a co-immunoprecipitation assay.

31. The method of claim 27, wherein said candidate substance is a polynucleotide, a polypeptide, or a small molecule inhibitor.

32. The method of claim 31, wherein said polynucleotide encodes, or said polypeptide is, an enzyme, an antibody, an antisense mRNA, or a transcription factor.

33. The method of claim 32, wherein said antibody reacts immunologically to said Rad51/Xrcc3 complex.

34. The method of claim 31, wherein said polynucleotide is an expression construct comprising a promoter active in eukaryotic cells.

35. The method of claim 27, wherein said candidate substance is selected from a small molecule or peptide library.

36. A method for preventing or treating cellular damage in an animal patient exposed to a DNA damaging agent comprising administering to said patient a pharmaceutically acceptable composition comprising Rad51 or Xrcc3.

37. The method of claim 36, wherein said Rad51 or said Xrcc3 is provided to said animal patient as a first polynucleotide encoding a Rad51 polypeptide or a second polynucleotide encoding a Xrcc3 polypeptide.

38. The method of claim 37, wherein said first or said second polynucleotide is delivered to said animal patient as naked DNA or through viral delivery.

39. The method of claim 37, wherein said polynucleotides are under the control of a promoter operatively linked to said first and said second polynucleotides, respectively.

40. The method of claim 39, wherein said promoter is a radiation-inducible promoter.

41. The method of claim 37, wherein said polynucleotides have a polyadenylation signal positioned 3′ to said first and said second polynucleotides, respectively.

42. The method of claim 37, wherein said Rad51 or said Xrcc3 is provided to said animal patient under the control of a selectable marker.

43. The method of claim 37, wherein said first or said second polynucleotide is contained in a viral vector.

44. The method of claim 36, wherein said Rad51 and said Xrcc3 are both provided to said animal patient as polynucleotides encoding a Rad51 polypeptide and a Xrcc3 polypeptide, respectively.

45. The method of claim 44, wherein said first and said second polynucleotides are delivered to said animal patient as naked DNA or through viral delivery.

46. The method of claim 44, wherein said first and said second polynucleotides are contained in viral vectors.

47. The method of claim 44, wherein both said Rad51 and said Xrcc3 are provided to said animal patient under the control of a selectable marker.

48. The method of claim 44, wherein said Rad51 and said Xrcc3 are provided to said animal patient as a Rad51/Xrcc3 protein complex.

49. The method of claim 36, wherein said animal patient is a human.

50. The method of claim 36, wherein said DNA damaging agent is radiation or a chemotherapeutic agent.

51. The method of claim 50, wherein said radiation is ionizing radiation or ultraviolet radiation.

52. The method of claim 50, wherein said chemotherapeutic agent is cisplatin, adriamycin, 5-fluorouracil, etoposide, camptothecin, actinomycin-D, verapamil or mitomycin C.

53. A method for treating an animal patient with cancer comprising contacting cancer cells in said patient with a pharmaceutically acceptable composition comprising Rad51 antisense RNA or Xrcc3 antisense RNA, wherein said antisense RNA blocks the formation of a functional Rad51/Xrcc3 complex.

54. The method of claim 53, comprising the additional step of contacting said cancer cells in said patient with a DNA damaging agent.

55. The method of claim 53, wherein said Rad51 antisense RNA or said Xrcc3 antisense RNA is provided to said animal patient as a first polynucleotide encoding a Rad51 antisense RNA or a second polynucleotide encoding a Xrcc3 antisense RNA.

56. The method of claim 55, wherein said first or said second polynucleotide is delivered to said animal patient as naked DNA or through viral delivery.

57. The method of claim 55, wherein said polynucleotides are under the control of a promoter operatively linked to said first and said second polynucleotides, respectively.

58. The method of claim 57, wherein said promoter is a radiation-inducible promoter.

59. The method of claim 55, wherein said polynucleotides have a polyadenylation signal positioned 3′ to said first and said second polynucleotides, respectively.

60. The method of claim 55, wherein said Rad51 antisense RNA or said Xrcc3 anti sense RNA is provided to said animal patient under the control of a selectable marker.

61. The method of claim 55, wherein said first or said second polynucleotide is contained in a viral vector.

62. The method of claim 36, wherein said Rad51 antisense RNA and said Xrcc3 antisense RNA are both provided to said animal patient as polynucleotides encoding a Rad51 antisense RNA and a Xrcc3 antisense RNA, respectively.

63. The method of claim 62, wherein said first and said second polynucleotides are delivered to said animal patient as naked DNA or through viral delivery.

64. The method of claim 62, wherein said first and said second polynucleotides are contained in viral vectors.

64. The method of claim 62, wherein both said Rad51 antisense RNA and said Xrcc3 antisense RNA are provided to said animal patient under the control of a selectable marker.

65. The method of claim 53, wherein said animal patient is a human.

66. The method of claim 54, wherein said DNA damaging agent is radiation or a chemotherapeutic agent.

67. The method of claim 66, wherein said radiation is ionizing radiation or ultraviolet radiation.

68. The method of claim 66, wherein said chemotherapeutic agent is cisplatin, adriamycin, 5-fluorouracil, etoposide, camptothecin, actinomycin-D, mitomycin C, verapamil or the DNA alkylating agent methylmethane sulfonate (MMS).
